SEQUENCE LISTING (17 sequences)

SEQ ID NO: 1 - DNA, 52 bp, Artificial Sequence
  Forward Primer - PL2_5BC_0N_B15
gtctcgtgggctcggagatgtgtataagagacagccgccagaacacagctag

SEQ ID NO: 2 - DNA, 53 bp, Artificial Sequence
  Forward Primer - PL2_5BC_1N_B15
  misc_feature (35)..(35): n is any nucleotide
gtctcgtgggctcggagatgtgtataagagacagnccgccagaacacagctag

SEQ ID NO: 3 - DNA, 54 bp, Artificial Sequence
  Forward Primer - PL2_5BC_2N_B15
  misc_feature (35)..(36): n is any nucleotide
gtctcgtgggctcggagatgtgtataagagacagnnccgccagaacacagctag

SEQ ID NO: 4 - DNA, 55 bp, Artificial Sequence
  Forward Primer - PL2_5BC_3N_B15
  misc_feature (35)..(37): n is any nucleotide
gtctcgtgggctcggagatgtgtataagagacagnnnccgccagaacacagctag

SEQ ID NO: 5 - DNA, 53 bp, Artificial Sequence
  Reverse primer
tcgtcggcagcgtcagatgtgtataagagacagccagaggttgattgtcgaca

SEQ ID NO: 6 - DNA, 33 bp, Artificial Sequence
  Reverse primer - A14-ME adaptor sequence
tcgtcggcagcgtcagatgtgtataagagacag

SEQ ID NO: 7 - DNA, 151 bp, Artificial Sequence
  Read1 for exogenous variant transcript - Cell barcode+UMI+(TSO)
ctacaccgtaattggaacatctgatcacccgtatatcaactttttcgcaacgggtttgccgccagaacacagctagcgacctgagctagggtcgcgcgtaagtgaccaacactctcgagcggccgccactgtgctggatatctgcagaatt

SEQ ID NO: 8 - DNA, 151 bp, Artificial Sequence
  Read1 for exogenous variant transcript - Cell barcode+UMI+(TSO)
gggatgacatccaacacccgtgttattatctaatatctggcttttttttcgcaacgggtttgccgccagaacacagctagcgtccacatcccaggtgcatcgtaagtgaccaacactctcgagcggccgccactgtgctggatatctgcag

SEQ ID NO: 9 - DNA, 151 bp, Artificial Sequence
  Read1 for exogenous variant transcript - Cell barcode+UMI+(TSO)
agcgtatcacagcccattcaatgataaaacaaatcacaagctttttcgcaacgggtttgccgccagaacacagctagccagctgatcccatgagaatcgtaagtgaccaacactctcgagcggccgccactgtgctggagatctgcagaat

SEQ ID NO: 10 - DNA, 151 bp, Artificial Sequence
  Read1 for exogenous variant transcript - Cell barcode+UMI+(TSO)
gtgaaggcagccagaagtatgtggatgcacttatatcgagcttttttcgcaacgggtttgccgccagaacacagctagccttcactttttaatttaatcgtaagtgaccaacactctcgagcggccgccactgtgctggatatctgcagaa

SEQ ID NO: 11 - DNA, 151 bp, Artificial Sequence
  Read1 for exogenous variant transcript - Cell barcode+UMI+(TSO)
ggtattggtactcgcgtttacattgctaactaatatcaagctttttcgcaacgggtttgccgccagaacacagctagcgttctcagtttgcttttatcgtaagtgaccaacactctcgagcggccgccactgtgctggatatctgcagaat

SEQ ID NO: 12 - DNA, 151 bp, Artificial Sequence
  Read1 for exogenous variant transcript - Cell barcode+UMI+(TSO)
cgaatgttcttaacctgacccccccccttcttataacatgctttttcgcaacgggtttgccgccagaacacagctagcgtgcggtgttaattacactcgtaagtgaccaacactctcgagcggccgccactgtgctggatatctgcagaat

SEQ ID NO: 13 - DNA, 151 bp, Artificial Sequence
  Read1 for exogenous variant transcript - Cell barcode+UMI+(TSO)
gtaactgagagactatcttctttatccttcttctatgaggctttttcgcaacgggtttgccgccagaacacagctagccaccagtcacgggggccctcgtaagtgaccaacactctcgagcggccgccactgtgctggatatctgcagaat

SEQ ID NO: 14 - DNA, 151 bp, Artificial Sequence
  Read1 for exogenous variant transcript - Cell barcode+UMI+(TSO)
ctgcctaagcgtgaacggaatgcgcgtttcttatatgttgctttttcgcaacgggtttgccgccagaacacagctagcgttcacttctggatatcctcgtaagtgaccaacactctcgagcggccgccactgtgctggatatctgcagaat

SEQ ID NO: 15 - DNA, 151 bp, Artificial Sequence
  Read1 for exogenous variant transcript - Cell barcode+UMI+(TSO)
gcacactagcgttccgggtacgggcagatcttatctggggctttttcgcaacgggtttgccgccagaacacagctagcctgctcaaattagtatcatcgtaagtgaccaacactctcgagcggccgccactgtgctggatatctgcagaat

SEQ ID NO: 16 - DNA, 151 bp, Artificial Sequence
  Read1 for exogenous variant transcript - Cell barcode+UMI+(TSO)
tttactgtcggaaatatcactccatatttcttatatggtgctttttcgcaacgggtttgccgccagaacacagctagccagctcagctcgatatgatcgtaagtgaccaacactctcgagcggccgccactgtgctgtatatctgcagaat

SEQ ID NO: 17 - DNA, 19 bp, Artificial Sequence
  example primer 4997F EFs
tgatgtcgtgtactggctc

(19) United States
(12) Patent Application Publication
(10) Pub. No.: US 2024/0167020 A1
(43) Pub. Date: May 23, 2024

(54) ANALYZING EXPRESSION OF PROTEIN-CODING VARIANTS IN CELLS

(71) Applicant: Illumina, Inc., San Diego, CA (US)

(72) Inventors: Hongxia Xu, Castro Valley, CA (US); Tong Liu, Belmont, CA (US); Shi Min Xiao, Castro Valley, CA (US); Dan Cao, East Palo Alto, CA (US); Victor Quijano, Mountain View, CA (US); Kai-How Farh, Hillsborough, CA (US); Mohan Sun, Albany, CA (US)

(73) Assignee: Illumina, Inc., San Diego, CA (US)

(21) Appl. No.: 18/549,343

(22) PCT Filed: Mar. 8, 2022
(86) PCT No.: PCT/US22/19258

§ 371 Date: Sep. 6, 2023

Related U.S. Application Data
(60) Provisional application No. 63/158,492, filed on Mar. 9, 2021.
(60) Provisional application No. 63/162,775, filed on Mar. 18, 2021.
(60) Provisional application No. 63/163,381, filed on Mar. 19, 2021.
(60) Provisional application No. 63/226,424, filed on Jul. 28, 2021.

(51) Int. Cl. C12N 15/10

(52) U.S. Cl. CPC C12N 15/1082; C12N 15/1062; C12N 15/1065

(57) ABSTRACT

Analyzing expression of protein-coding variants in cells is provided herein. A method may include replacing a protein coding-region of the DNA in a cell with a donor vector including a variant of the protein-coding region and a first barcode identifying that variant. The cell may generate mRNA including an expression of the variant and an expression of the first barcode. A second barcode corresponding to the cell may be coupled to the mRNA. The mRNA. having the second barcode coupled thereto, may be reverse transcribed into complementary cDNA. The cDNA may be sequenced. The donor vector or cDNA may be sequenced using amplicon sequencing. The donor vector sequence and the cDNA sequence may be correlated to identify the variant and the cell's expression of the variant.

### CROSS-REFERENCE TO RELATED APPLICATIONS

This application claims the benefit of the following applications, the entire contents of each of which are incorporated by reference herein:
U.S. Provisional Patent Application No. 63/158,492, filed Mar. 9, 2021 and entitled “Genomic library preparation and targeted epigenetic assays using Cas-gRNA ribonucleoproteins;”
U.S. Provisional Patent Application No. 63/162,775, filed Mar. 18, 2021 and entitled “Genomic library preparation and targeted epigenetic assays using Cas-gRNA ribonucleoproteins;”
U.S. Provisional Patent Application No. 63/163,381, filed Mar. 19, 2021 and entitled “Genomic library preparation and targeted epigenetic assays using Cas-gRNA ribonucleoproteins;” and
U.S. Provisional Patent Application No. 63/226,424, filed Jul. 28, 2021 and entitled “Analyzing Expression of Protein-Coding Variants in Cells.”

### FIELD

This application relates to compositions and methods for analyzing protein-coding variants in cells.

### STATEMENT REGARDING SEQUENCE LISTING

The Sequence Listing associated with this application is provided in text format in lieu of a paper copy, and is hereby incorporated by reference into the specification. The name of the text file containing the Sequence Listing is 8549103716_SL.txt. The text file is about 5.73 KB, was created on Feb. 18, 2022, and is being submitted electronically via EFS-Web.

### BACKGROUND

Specific phenotypic assays have been used to attempt to determine the function of variants or the effects of genome editing, but are low throughput. For example, such assays may provide information that pertains to the function of just a single variant or edit, and may not provide information that pertains to the functions of any other variants. Single-cell RNA sequencing (scRNA-seq) is commercially available and may be used to obtain and sequence the transcriptome from a cell.

### SUMMARY

Analyzing expression of protein-coding variants in cells is provided herein.

Some examples herein provide a method of analyzing expression of a protein-coding region of DNA in a cell. The method may include replacing a protein-coding region of the DNA in the cell with a donor vector including a variant of the protein-coding region and a first barcode identifying that variant. The cell may generate mRNA including an expression of the variant and an expression of the first barcode. The method may include coupling, to the mRNA, a second barcode corresponding to the cell. The method may include reverse transcribing the mRNA, having the second barcode coupled thereto, into cDNA. The method may include sequencing the cDNA. The method may include sequencing the donor vector or cDNA using amplicon sequencing. The method may include correlating the donor vector sequence and the cDNA sequence to identify the variant and the cell's expression of the variant.

In some examples, the donor vector includes a promoter region. In some examples, the barcode is located between the promoter region and the variant. In some examples, the donor vector includes right and left homology arms, the variant and the first barcode being between the right and left homology arms. In some examples, the promoter region includes a reverse promotor region. In some examples, the reverse promoter region is disposed between the first barcode and the variant. In some examples, the expression of the variant of the protein-coding region is in the forward direction, and wherein the expression of the first barcode is in the reverse direction.

Additionally, or alternatively, in some examples, the method further includes using a first polymerase chain reaction (PCR) process to generate a first amplicon of the donor sequence that includes the variant, the first barcode, and the right homology arm and substantially excludes the left homology arm. The method may include using a second PCR process to generate a second amplicon of the first amplicon that includes the variant and the first barcode and substantially excludes the right and left homology arms. Additionally, or alternatively, in some examples, sequencing the donor vector includes sequencing the second amplicon. Additionally, or alternatively, in some examples, the second amplicon has a length of about 1000 bases or fewer.

Additionally, or alternatively, in some examples, the mRNA includes a first mRNA molecule including the expression of the variant, and a second mRNA molecule including the expression of the first barcode. In some examples, coupling the second barcode to the mRNA includes coupling a first molecule of the second barcode to the first mRNA molecule: and coupling a second molecule of the second barcode to the second mRNA molecule. Additionally, or alternatively, in some examples, the cDNA includes a first cDNA molecule including a reverse transcription of the variant and the second barcode, and a second cDNA molecule including a reverse transcription of the protein coding region and the second barcode, and sequencing the cDNA includes sequencing the first and second cDNA molecules.

Additionally, or alternatively, in some examples, replacing the initial protein-coding region includes using a CRISPR-associated protein guide RNA ribonucleoprotein (Cas-gRNA RNP) to cut the DNA in the cell; and using homology-directed repair (HDR) to repair the cut in the DNA using the donor vector. In some examples, the method further includes inserting first and second plasmids into the cell. The donor vector may be located on the first plasmid. The cell may express the Cas-gRNA RNP using the second plasmid.

Additionally, or alternatively, in some examples, the donor vector includes a lentiviral vector.

Additionally, or alternatively, in some examples, the donor vector further includes a puromycin resistance gene, the method further including contacting the cell with puromycin to enrich for the cell. In some examples, the first barcode is located on a UTR terminus of the puromycin resistance gene.

Additionally, or alternatively, in some examples, the method further includes cleaving the first barcode from the variant in the cell.

Some examples herein provide a method of analyzing expression of a protein-coding region of DNA in a collection of cells. The method may include replacing the initial protein coding-region of the DNA in each of the cells with a donor vector including a variant of the protein-coding region and a first barcode identifying that variant. The cells may receive different variants than one another. The method may include obtaining mRNA from the cells. The mRNA from each cell may include an expression of the variant of the protein-coding region in that cell and an expression of the first barcode. The method may include coupling, to the mRNA from each cell, a second barcode corresponding to that cell. The method may include reverse transcribing the mRNA, having the second barcode coupled thereto, into cDNA. The method may include sequencing the cDNA. The method may include sequencing the donor vector or cDNA using amplicon sequencing. The method may include correlating the donor vector sequence and the cDNA sequence to identify the variant in each of the cells and that cell's expression of that variant.

In some examples, the different variants are saturationally mutagenized.

Some examples herein provide a collection of cells. The DNA of each of the cells in the collection may include a variant of a protein-coding region and a first barcode identifying that variant. The cells may have different variants than one another.

In some examples, the different variants are saturationally mutagenized.

Some examples herein provide a collection of polynucleotides from a collection of cells. The polynucleotides may include first and second mRNA molecules from each of the cells. For each cell, the first mRNA molecule includes a first molecule of a barcode corresponding to that cell and an expression of a variant in that cell, and the second mRNA molecule includes the barcode corresponding to that cell and an expression of a first barcode corresponding to the variant.

In some examples, the different variants are saturationally mutagenized.

Some examples herein provide a method. The method may include providing a barcoded homology donor vector including a semi-random barcode on termini of a foreign transcript. The donor vector may include homology arms and mutations. The method may include knocking-in the barcoded homology donor vector to the vicinity of an exon to be edited to create a variant on the exon. The method may include cleaving the variant using a CRISPR-associated protein guide RNA ribonucleoprotein (Cas-gRNA RNP).

In some examples, the barcode is placed on UTR termini of the donor vector so that it may be expressed and detectable in scRNA-seq.

In some examples, the donor vector includes a puromycin resistance gene.

In some examples, providing the barcoded homology donor vector may include using a first polymerase chain reaction (PCR) to specifically amplify the knocked-in region with a genomically edited allele: using a second PCR, using the product of the first PCR as a template, to link the barcode with variants in an amplicon; and performing amplicon sequencing using the product from the second PCR.

In some examples, the amplicon sequencing covers both the barcode and the variants.

Some examples herein provide a method. The method may include adding semi-random variant barcodes to UTR regions of a saturationally mutagenized variant library. The method may include coupling cell barcodes to the variant barcodes. The method may include reading the variant barcodes out in scRNA-seq. The method may include linking the variant barcodes to the variants of the library using a separate sequencing operation.

In some examples, the semi-random variant barcode may be placed downstream of promoters or upstream of terminators of the variant library.

In some examples, linking the variant barcodes to the variants of the library may include generating tiled polymerase chain reaction (PCR) amplicons by using one set of primers to amplify the barcode on one side, and another set of primers to amplify the variants on the other side, such that each amplicon links a respective segment of the variant to the barcode.

Some examples herein provide a lentiviral vector including a semi-random barcode.

Some examples herein provide a composition that includes a plurality of lentiviral vectors, each of the lentiviral vectors including a different semi-random barcode.

In some examples, the composition further includes a mutagenically saturated variant library in contact with the plurality of lentiviral vectors.

It is to be understood that any respective features/examples of each of the aspects of the disclosure as described herein may be implemented together in any appropriate combination, and that any features/examples from any one or more of these aspects may be implemented together with any of the features of the other aspect(s) as described herein in any appropriate combination to achieve the benefits as described herein.

### BRIEF DESCRIPTION OF THE DRAWINGS

FIGS. 1A-1E schematically illustrate example compositions and operations in a process flow for analyzing expression of protein-coding variants in cells.

FIG. 2 illustrates a flow of operations in an example method for analyzing expression of protein-coding variants in cells.

FIGS. 3A-3C schematically illustrate example compositions and operations in a process flow for random barcoded saturation genome editing for a high throughput protein coding variant assay by single cell RNA-seq (scRNA-seq).

FIGS. 4A-4E schematically illustrate example compositions and operations in a process flow for a high throughput protein coding variant assay by single cell RNA-seq (scRNA-seq) using an exogenous variant library that is saturationally mutagenized.

FIG. 5 depicts next generation sequencing results of amplicons that were PCR-amplified from edited genomes derived from a saturation genome experiment that targeted exon 7 of TP53.

FIG. 6 depicts a lentiviral based library with scRNA-seq as the readout.

### DETAILED DESCRIPTION

Analyzing expression of protein-coding variants in cells is provided herein.

Some examples herein relate to libraries of barcoded, protein-coding variants. The variants of the library may be introduced into respective cells, and single-cell RNA sequencing (scRNA-seq) used to analyze the cells' respective expression of each variant. In parallel, DNA sequencing may be used to sequence the variants. Different barcodes may be used to correlate the DNA sequence of each variant with the corresponding cell's expression of the variant as measured by scRNA-seq. In some examples, the barcoded variants in the library may be saturationally mutagenized, such that every base in the coding region for a protein may be mutagenized to the three other alternative bases, thereby generating up to nine different amino acids or stop codons for each codon. Therefore, the expression resulting from every possible variant on the coding region of a gene may be analyzed. However, it will be appreciated that any suitably genomically edited variant may be introduced, and the resulting expression analyzed. Regardless of the particular type of barcoded variants used in the library, scRNA-seq and DNA sequencing may be used synergistically to analyze the cells' expression of those variants in a scalable, highly multiplexed, and high throughput manner.

First, some terms used herein will be briefly explained. Then, some example operations and compositions for generating and assaying libraries of protein-coding variants will be described.

### Terms

Unless defined otherwise, all technical and scientific terms used herein have the same meaning as is commonly understood by one of ordinary skill in the art. The use of the term “including” as well as other forms, such as “include,” “includes,” and “included,” is not limiting. The use of the term “having” as well as other forms, such as “have,” “has,” and “had,” is not limiting. As used in this specification, whether in a transitional phrase or in the body of the claim, the terms “comprise(s)” and “comprising” are to be interpreted as having an open-ended meaning. That is, the above terms are to be interpreted synonymously with the phrases “having at least” or “including at least.” For example, when used in the context of a process, the term “comprising” means that the process includes at least the recited steps, but may include additional steps. When used in the context of a compound, composition, or device, the term “comprising” means that the compound, composition, or device includes at least the recited features or components, but may also include additional features or components.

As used herein, the singular forms “a”, “an” and “the” include plural referents unless the content clearly dictates otherwise.

The terms “substantially,” “approximately,” and “about” used throughout this specification are used to describe and account for small fluctuations, such as due to variations in processing. For example, they may refer to less than or equal to +10%, such as less than or equal to +5%, such as less than or equal to +2%, such as less than or equal to #1%, such as less than or equal to +0.5%, such as less than or equal to +0.2%, such as less than or equal to +0.1%, such as less than or equal to =0.05%.

As used herein, terms such as “hybridize” and “hybridization” are intended to mean noncovalently associating a polynucleotides to one another along the lengths of those polynucleotides to form a double-stranded “duplex,” a three-stranded “triplex,” or higher-order structure For example, two DNA polynucleotide strands may associate through complementary base pairing to form a duplex. The primary interaction between polynucleotide strands typically is nucleotide base specific, e.g., A:T, A:U, and G:C, by Watson-Crick and Hoogsteen-type hydrogen bonding. Base-stacking and hydrophobic interactions also may contribute to duplex stability. Hybridization conditions may include salt concentrations of less than about 1 M, more usually less than about 500 mM, or less than about 200 mM. A hybridization buffer may include a buffered salt solution such as 5% SSPE or other suitable buffer known in the art. Hybridization temperatures may be as low as 5° C., but are typically greater than 22° C., and more typically greater than about 30° C., and typically in excess of 37° C. The strength of the association between the first and second polynucleotides increases with the complementarity between the sequences of nucleotides within those polynucleotides. The strength of hybridization between polynucleotides may be characterized by a temperature of melting (Tm) at which 50% of the duplexes have polynucleotide strands that disassociate from one another.

As used herein, the term “nucleotide” is intended to mean a molecule that includes a sugar and at least one phosphate group, and in some examples also includes a nucleobase. A nucleotide that lacks a nucleobase may be referred to as “abasic.” Nucleotides include deoxyribonucleotides, modified deoxyribonucleotides, ribonucleotides, modified ribonucleotides, peptide nucleotides, modified peptide nucleotides, modified phosphate sugar backbone nucleotides, and mixtures thereof. Examples of nucleotides include adenosine monophosphate (AMP), adenosine diphosphate (ADP), adenosine triphosphate (ATP), thymidine monophosphate (TMP), thymidine diphosphate (TDP), thymidine triphosphate (TTP), cytidine monophosphate (CMP), cytidine diphosphate (CDP), cytidine triphosphate (CTP), guanosine monophosphate (GMP), guanosine diphosphate (GDP), guanosine triphosphate (GTP), uridine monophosphate (UMP), uridine diphosphate (UDP), uridine triphosphate (UTP), deoxyadenosine monophosphate (dAMP), deoxyadenosine diphosphate (dADP), deoxyadenosine triphosphate (dATP), deoxythymidine monophosphate (dTMP), deoxy thymidine diphosphate (dTDP), deoxythymidine triphosphate (dTTP), deoxycytidine diphosphate (dCDP), deoxycytidine triphosphate (dCTP), deoxyguanosine monophosphate (dGMP), deoxyguanosine diphosphate (dGDP), deoxyguanosine triphosphate (dGTP), deoxyuridine monophosphate (dUMP), deoxyuridine diphosphate (dUDP), and deoxyuridine triphosphate (dUTP).

As used herein, the term “nucleotide” also is intended to encompass any nucleotide analogue which is a type of nucleotide that includes a modified nucleobase, sugar, backbone, and/or phosphate moiety compared to naturally occurring nucleotides. Nucleotide analogues also may be referred to as “modified nucleic acids.” Example modified nucleobases include inosine, xathanine, hypoxathanine, isocytosine, isoguanine, 2-aminopurine, 5-methylcytosine, 5-hydroxymethyl cytosine, 2-aminoadenine, 6-methyl adenine, 6-methyl guanine, 2-propyl guanine, 2-propyl adenine, 2-thiouracil, 2-thiothymine, 2-thiocytosine, 15-halouracil, 15-halocytosine, 5-propynyl uracil, 5-propynyl cytosine, 6-azo uracil, 6-azo cytosine, 6-azo thymine, 5-uracil, 4-thiouracil, 8-halo adenine or guanine, 8-amino adenine or guanine, 8-thiol adenine or guanine, 8-thioalkyl adenine or guanine, 8-hydroxyl adenine or guanine, 5-halo substituted uracil or cytosine, 7-methylguanine, 7-methyladenine, 8-azaguanine, 8-azaadenine, 7-deazaguanine, 7-deazaadenine, 3-deazaguanine, 3-deazaadenine or the like. As is known in the art, certain nucleotide analogues cannot become incorporated into a polynucleotide, for example, nucleotide analogues such as adenosine 5′-phosphosulfate. Nucleotides may include any suitable number of phosphates, e.g., three, four, five, six, or more than six phosphates. Nucleotide analogues also include locked nucleic acids (LNA), peptide nucleic acids (PNA), and 5-hydroxylbutynl-2′-deoxyuridine (“super T”).

As used herein, the term “polynucleotide” refers to a molecule that includes a sequence of nucleotides that are bonded to one another. A polynucleotide is one nonlimiting example of a polymer. Examples of polynucleotides include deoxyribonucleic acid (DNA), ribonucleic acid (RNA), and analogues thereof such as locked nucleic acids (LNA) and peptide nucleic acids (PNA). A polynucleotide may be a single stranded sequence of nucleotides, such as RNA or single stranded DNA, a double stranded sequence of nucleotides, such as double stranded DNA, or may include a mixture of a single stranded and double stranded sequences of nucleotides. Double stranded DNA (dsDNA) includes genomic DNA, and PCR and amplification products. Single stranded DNA (ssDNA) can be converted to dsDNA and vice-versa. Polynucleotides may include non-naturally occurring DNA, such as enantiomeric DNA, LNA, or PNA. The precise sequence of nucleotides in a polynucleotide may be known or unknown. The following are examples of polynucleotides: a gene or gene fragment (for example, a probe, primer, expressed sequence tag (EST) or serial analysis of gene expression (SAGE) tag), genomic DNA, genomic DNA fragment, exon, intron, messenger RNA (mRNA), transfer RNA, ribosomal RNA, ribozyme, cDNA, recombinant polynucleotide, synthetic polynucleotide, branched polynucleotide, plasmid, vector, isolated DNA of any sequence, isolated RNA of any sequence, nucleic acid probe, primer or amplified copy of any of the foregoing.

As used herein, a “polymerase” is intended to mean an enzyme having an active site that assembles polynucleotides by polymerizing nucleotides into polynucleotides. A polymerase can bind a primed single stranded target polynucleotide, and can sequentially add nucleotides to the growing primer to form a “complementary copy” polynucleotide having a sequence that is complementary to that of the target polynucleotide. Another polymerase, or the same polymerase, then can form a copy of the target nucleotide by forming a complementary copy of that complementary copy polynucleotide. DNA polymerases may bind to the target polynucleotide and then move down the target polynucleotide sequentially adding nucleotides to the free hydroxyl group at the 3′ end of a growing polynucleotide strand (growing amplicon). DNA polymerases may synthesize complementary DNA molecules from DNA templates and RNA polymerases may synthesize RNA molecules from DNA templates (transcription). Polymerases may use a short RNA or DNA strand (primer), to begin strand growth. Some polymerases may displace the strand upstream of the site where they are adding bases to a chain. Such polymerases may be said to be strand displacing, meaning they have an activity that removes a complementary strand from a template strand being read by the polymerase.

Example polymerases include Bst DNA polymerase, 9° Nm DNA polymerase, Phi29 DNA polymerase, DNA polymerase I (E. coli), DNA polymerase I (Large), (Klenow) fragment, Klenow fragment (3′-5′ exo-), T4 DNA polymerase, T7 DNA polymerase, Deep VentR™ (exo-) DNA polymerase, Deep VentR™ DNA polymerase, DyNAzyme™ EXT DNA, DyNAzyme™ II Hot Start DNA Polymerase, Phusion™ High-Fidelity DNA Polymerase, Therminator™ DNA Polymerase, Therminator™ II DNA Polymerase, VentR®; DNA Polymerase, VentR® (exo-) DNA Polymerase, RepliPHI™ Phi29 DNA Polymerase, rBst DNA Polymerase, rBst DNA Polymerase (Large), Fragment (IsoTherm™ DNA Polymerase), MasterAmp™ AmpliTherm™, DNA Polymerase, Taq DNA polymerase, Tth DNA polymerase, Tfl DNA polymerase, Tgo DNA polymerase, SP6 DNA polymerase, Tbr DNA polymerase, DNA polymerase Beta, and ThermoPhi DNA polymerase. In specific, nonlimiting examples, the polymerase is selected from a group consisting of Bst, Bsu, and Phi29. As the polymerase extends the hybridized strand, it can be beneficial to include single-stranded binding protein (SSB). SSB may stabilize the displaced (non-template) strand. Example polymerases having strand displacing activity include, without limitation, the large fragment of Bst (Bacillus stearothermophilus) polymerase, exo-Klenow polymerase or sequencing grade T7 exo-polymerase. Some polymerases degrade the strand in front of them, effectively replacing it with the growing chain behind (5′ exonuclease activity). Some polymerases have an activity that degrades the strand behind them (3′ exonuclease activity). Some useful polymerases have been modified, either by mutation or otherwise, to reduce or eliminate 3′ and/or 5′ exonuclease activity.

As used herein, the term “primer” is defined as a polynucleotide to which nucleotides may be added via a free 3′ OH group. A primer may include a 3′ block inhibiting polymerization until the block is removed. A primer may include a modification at the 5′ terminus to allow a coupling reaction or to couple the primer to another moiety. A primer may include one or more moieties, such as 8-oxo-G, which may be cleaved under suitable conditions, such as UV light, chemistry, enzyme, or the like. The primer length may be any suitable number of bases long and may include any suitable combination of natural and non-natural nucleotides. A target polynucleotide may include an “amplification adapter” or, more simply, an “adapter,” that hybridizes to (has a sequence that is complementary to) a primer, and may be amplified so as to generate a complementary copy polynucleotide by adding nucleotides to the free 3′ OH group of the primer.

As used herein, the term “plurality” is intended to mean a population of two or more different members. Pluralities may range in size from small, medium, large, to very large. The size of small plurality may range, for example, from a few members to tens of members. Medium sized pluralities may range, for example, from tens of members to about 100 members or hundreds of members. Large pluralities may range, for example, from about hundreds of members to about 1000 members, to thousands of members and up to tens of thousands of members. Very large pluralities may range, for example, from tens of thousands of members to about hundreds of thousands, a million, millions, tens of millions and up to or greater than hundreds of millions of members. Therefore, a plurality may range in size from two to well over one hundred million members as well as all sizes, as measured by the number of members, in between and greater than the above example ranges. Example polynucleotide pluralities include, for example, populations of about 1×10⁵ or more, 5×10⁵ or more, or 1×10⁶ or more different polynucleotides. Accordingly, the definition of the term is intended to include all integer values greater than two. An upper limit of a plurality may be set, for example, by the theoretical diversity of polynucleotide sequences in a sample.

As used herein, the term “double-stranded,” when used in reference to a polynucleotide, is intended to mean that all or substantially all of the nucleotides in the polynucleotide are hydrogen bonded to respective nucleotides in a complementary polynucleotide. A double-stranded polynucleotide also may be referred to as a “duplex.” As used herein, the term “single-stranded,” when used in reference to a polynucleotide, means that essentially none of the nucleotides in the polynucleotide are hydrogen bonded to a respective nucleotide in a complementary polynucleotide.

As used herein, the term “target polynucleotide” is intended to mean a polynucleotide that is the object of an analysis or action. The analysis or action includes subjecting the polynucleotide to capture, amplification, sequencing and/or other procedure. A target polynucleotide may include nucleotide sequences additional to a target sequence to be analyzed. For example, a target polynucleotide may include one or more adapters, including an amplification adapter that functions as a primer binding site, that flank(s) a target polynucleotide sequence that is to be analyzed. A target polynucleotide hybridized to a primer may include nucleotides that extend beyond the 5′ or 3′ end of the oligonucleotide in such a way that not all of the target polynucleotide is amenable to extension. In particular examples, target polynucleotides may have different sequences than one another but may have first and second adapters that are the same as one another. The two adapters that may flank a particular target polynucleotide sequence may have the same sequence as one another, or complementary sequences to one another, or the two adapters may have different sequences. Thus, species in a plurality of target polynucleotides may include regions of known sequence that flank regions of unknown sequence that are to be evaluated by, for example, sequencing (e.g., SBS). In some examples, target polynucleotides carry an amplification adapter at a single end, and such adapter may be located at either the 3′ end or the 5′ end the target polynucleotide. Target polynucleotides may be used without any adapter, in which case a primer binding sequence may come directly from a sequence found in the target polynucleotide.

The terms “polynucleotide” and “oligonucleotide” are used interchangeably herein. The different terms are not intended to denote any particular difference in size, sequence, or other property unless specifically indicated otherwise. For clarity of description, the terms may be used to distinguish one species of polynucleotide from another when describing a particular method or composition that includes several polynucleotide species.

The terms “sequence” and “subsequence” may in some cases be used interchangeably herein. For example, a sequence may include one or more subsequences therein. Each of such subsequences also may be referred to as a sequence.

As used herein, the term “amplicon,” when used in reference to a polynucleotide, is intended to mean a product of copying the polynucleotide, wherein the product has a nucleotide sequence that is substantially the same as, or is substantially complementary to, at least a portion of the nucleotide sequence of the polynucleotide. “Amplification” and “amplifying” refer to the process of making an amplicon of a polynucleotide. A first amplicon of a target polynucleotide may be a complementary copy. Additional amplicons are copies that are created, after generation of the first amplicon, from the target polynucleotide or from the first amplicon. A subsequent amplicon may have a sequence that is substantially complementary to the target polynucleotide or is substantially identical to the target polynucleotide. It will be understood that a small number of mutations (e.g., due to amplification artifacts) of a polynucleotide may occur when generating an amplicon of that polynucleotide.

As used herein, terms such as “CRISPR-Cas system,” “Cas-gRNA ribonucleoprotein,” and Cas-gRNA RNP refer to an enzyme system including a guide RNA (gRNA) sequence that includes an oligonucleotide sequence that is complementary or substantially complementary to a sequence within a target polynucleotide, and a Cas protein. CRISPR-Cas systems may generally be categorized into three major types which are further subdivided into ten subtypes, based on core element content and sequences; see, e.g., Makarova et al., “Evolution and classification of the CRISPR-Cas systems,” Nat Rev Microbiol. 9(6): 467-477 (2011). Cas proteins may have various activities, e.g., nuclease activity. Thus, CRISPR-Cas systems provide mechanisms for targeting a specific sequence (e.g., via the gRNA) as well as certain enzyme activities upon the sequence (e.g., via the Cas protein).

A Type I CRISPR-Cas system may include Cas3 protein with separate helicase and DNase activities. For example, in the Type 1-E system, crRNAs are incorporated into a multisubunit effector complex called Cascade (CRISPR-associated complex for antiviral defense), which binds to the target DNA and triggers degradation by the Cas3 protein; see, e.g., Brouns et al., “Small CRISPR RNAs guide antiviral defense in prokaryotes,” Science 321(5891): 960-964 (2008); Sinkunas et al., “Cas3 is a single-stranded DNA nuclease and ATP-dependent helicase in the CRISPR-Cas immune system,” EMBO J 30: 1335-1342 (2011); and Beloglazova et al., “Structure and activity of the Cas3 HD nuclease MJ0384, an effector enzyme of the CRISPR interference, EMBO J 30:4616-4627 (2011). Type II CRISPR-Cas systems include the signature Cas9 protein, a single protein (about 160 KDa) capable of generating crRNA and cleaving the target DNA. The Cas9 protein typically includes two nuclease domains, a RuvC-like nuclease domain near the amino terminus and the HNH (or McrA-like) nuclease domain near the middle of the protein. Each nuclease domain of the Cas9 protein is specialized for cutting one strand of the double helix; see, e.g., Jinek et al., “A programmable dual-RNA-guided DNA endonuclease in adaptive bacterial immunity, Science 337(6096): 816-821 (2012). Type III CRISPR-Cas systems include polymerase and RAMP modules. Type III systems can be further divided into sub-types III-A and III-B. Type III-A CRISPR-Cas systems have been shown to target plasmids, and the polymerase-like proteins of Type III-A systems are involved in the cleavage of target DNA; see, e.g., Marraffini et al., “CRISPR interference limits horizontal gene transfer in Staphylococci by targeting DNA,” Science 322(5909): 1843-1845 (2008). Type III-B CRISPR-Cas systems have also been shown to target RNA; see, e.g., Hale et al., “RNA-guided RNA cleavage by a CRISPR-RNA-Cas protein complex,” Cell 139(5): 945-956 (2009). CRISPR-Cas systems include engineered and/or programmed nuclease systems derived from naturally accruing CRISPR-Cas systems. CRISPR-Cas systems may include engineered and/or mutated Cas proteins. CRISPR-Cas systems may include engineered and/or programmed guide RNA.

In some specific examples, the Cas protein in one of the present Cas-gRNA RNPs may include Cas9 or other suitable Cas that may cut the target polynucleotide at the sequence to which the gRNA is complementary, in a manner such as described in the following references, the entire contents of each of which are incorporated by reference herein: Nachmanson et al., “Targeted genome fragmentation with CRISPR/Cas9 enables fast and efficient enrichment of small genomic regions and ultra-accurate sequencing with low DNA input (CRISPR-DS),” Genome Res. 28(10): 1589-1599 (2018): Vakulskas et al., “A high-fidelity Cas9) mutant delivered as a ribonucleoprotein complex enables efficient gene editing in human hematopoietic stem and progenitor cells,” Nature Medicine 24: 1216-1224 (2018); Chatterjee et al., “Minimal PAM specificity of a highly similar SpCas9 ortholog,” Science Advances 4(10): eaau0766, 1-10 (2018); Lee et al., “CRISPR-Cap: multiplexed double-stranded DNA enrichment based on the CRISPR system,” Nucleic Acids Research 47(1): 1-13 (2019). Isolated Cas9-crRNA complex from the S. thermophilus CRISPR-Cas system as well as complex assembled in vitro from separate components demonstrate that it binds to both synthetic oligodeoxynucleotide and plasmid DNA bearing a nucleotide sequence complementary to the crRNA. It has been shown that Cas9 has two nuclease domains RuvC- and HNH-active sites/nuclease domains, and these two nuclease domains are responsible for the cleavage of opposite DNA strands. In some examples, the Cas9 protein is derived from Cas9 protein of S. thermophilus CRISPR-Cas system. In some examples, the Cas9 protein is a multi-domain protein having about 1,409 amino acids residues.

In other examples, the Cas may be engineered so as not to cut the target polynucleotide at the sequence to which the gRNA is complementary, e.g., in a manner such as described in the following references, the entire contents of each of which are incorporated by reference herein: Guilinger et al., “Fusion of catalytically inactive Cas9 to Fokl nuclease improves the specificity of genome modification,” Nature Biotechnology 32: 577-582 (2014); Bhatt et al., “Targeted DNA transposition using a dCas9-transposase fusion protein,” https://doi.org/10.1101/571653, pages 1-89 (2019); Xu et al., “CRISPR-assisted targeted enrichment-sequencing (CATE-seq),” available at URL www.biorxiv.org/content/10.1101/672816v1, 1-30 (2019); and Tijan et al., “dCas9-targeted locus-specific protein isolation method identifies histone gene regulators,” PNAS 115(12): E2734-E2741 (2018). Cas that lacks nuclease activity may be referred to as deactivated Cas (dCas). In some examples, the dCas may include a nuclease-null variant of the Cas9 protein, in which both RuvC- and HNH-active sites/nuclease domains are mutated. A nuclease-null variant of the Cas9) protein (dCas9) binds to double-stranded DNA, but does not cleave the DNA. Another variant of the Cas9 protein has two inactivated nuclease domains with a first mutation in the domain that cleaves the strand complementary to the crRNA and a second mutation in the domain that cleaves the strand non-complementary to the crRNA. In some examples, the Cas) protein has a first mutation D10A and a second mutation H840A.

In still other examples, the Cas protein includes a Cascade protein. Cascade complex in E. coli recognizes double-stranded DNA (dsDNA) targets in a sequence-specific manner. E. coli Cascade complex is a 405-kDa complex including five functionally essential CRISPR-associated (Cas) proteins (CasA1B2C6DIE1, also called Cascade protein) and a 61-nucleotide crRNA. The crRNA guides Cascade complex to dsDNA target sequences by forming base pairs with the complementary DNA strand while displacing the noncomplementary strand to form an R-loop. Cascade recognizes target DNA without consuming ATP, which suggests that continuous invader DNA surveillance takes place without energy investment; see, e.g., Matthijs et al., “Structural basis for CRISPR RNA-guided DNA recognition by Cascade,” Nature Structural & Molecular Biology 18(5): 529-536 (2011). In still other examples, the Cas protein includes a Cas3 protein. Illustratively, E. coli Cas3 may catalyze ATP-independent annealing of RNA with DNA forming R-loops, and hybrid of RNA base-paired into duplex DNA. Cas3 protein may use gRNA that is longer than that for Cas9); see, e.g., Howard et al., “Helicase disassociation and annealing of RNA-DNA hybrids by Escherichia coli Cas3 protein,” Biochem J. 439(1): 85-95 (2011). Such longer gRNA may permit easier access of other elements to the target DNA, e.g., access of a primer to be extended by polymerase. Another feature provided by Cas3 protein is that Cas3 protein does not require a PAM sequence as may Cas9, and thus provides more flexibility for targeting desired sequence. R-loop formation by Cas3 may utilize magnesium as a co-factor; see, e.g., Howard et al., “Helicase disassociation and annealing of RNA-DNA hybrids by Escherichia coli Cas3 protein,” Biochem J. 439(1): 85-95 (2011). It will be appreciated that any suitable cofactors, such as cations, may be used together with the Cas proteins used in the present compositions and methods.

It also should be appreciated that any CRISPR-Cas systems capable of disrupting the double stranded polynucleotide and creating a loop structure may be used. For example, the Cas proteins may include, but not limited to, Cas proteins such as described in the following references, the entire contents of each of which are incorporated by reference herein: Haft et al., “A guild of 45 CRISPR-associated (Cas) protein families and multiple CRISPR/Cas subtypes exist in prokaryotic genomes.” PLOS Comput Biol. 1(6): e60, 1-10 (2005): Zhang et al., “Expanding the catalog of cas genes with metagenomes,” Nucl. Acids Res. 42(4): 2448-2459 (2013); and Strecker et al., “RNA-guided DNA insertion with CRISPR-associated transposases,” Science 365(6448): 48-53 (2019) in which the Cas protein may include CasK12. Some these CRISPR-Cas systems may utilize a specific sequence to recognize and bind to the target sequence. For example, Cas9 may utilize the presence of a 5′-NGG protospacer-adjacent motif (PAM).

CRISPR-Cas systems may also include engineered and/or programmed guide RNA (gRNA). As used herein, the terms “guide RNA” and “gRNA” (and sometimes referred to in the art as single guide RNA, or sgRNA) is intended to mean RNA including a sequence that is complementary or substantially complementary to a region of a target DNA sequence and that guides a Cas protein to that region. A guide RNA may include nucleotide sequences in addition to that which is complementary or substantially complementary to the region of a target DNA sequence. Methods for designing gRNA are well known in the art, and nonlimiting examples are provided in the following references, the entire contents of each of which are incorporated by reference herein: Stevens et al., “A novel CRISPR/Cas9 associated technology for sequence-specific nucleic acid enrichment,” PLOS ONE 14(4): e0215441, pages 1-7 (2019): Fu et al., “Improving CRISPR-Cas nuclease specificity using truncated guide RNAs, Nature Biotechnology 32(3): 279-284 (2014); Kocak et al., “Increasing the specificity of CRISPR systems with engineered RNA secondary structures,” Nature Biotechnology 37: 657-666 (2019); Lee et al., “CRISPR-Cap: multiplexed double-stranded DNA enrichment based on the CRISPR system,” Nucleic Acids Research 47(1): el, 1-13 (2019); Quan et al., “FLASH: a next-generation CRISPR diagnostic for multiplexed detection of antimicrobial resistance sequences,” Nucleic Acids Research 47(14): e83, 1-9 (2019); and Xu et al., “CRISPR-assisted targeted enrichment-sequencing (CATE-seq),” https://doi.org/10.1101/672816, 1-30 (2019).

In some examples, gRNA includes a chimera, e.g., CRISPR RNA (crRNA) fused to trans-activating CRISPR RNA (tracrRNA). Such a chimeric single-guided RNA (sgRNA) is described in Jinek et al., “A programmable dual-RNA-guided endonuclease in adaptive bacterial immunity,” Science 337 (6096): 816-821 (2012). The Cas protein may be directed by a chimeric sgRNA to any genomic locus followed by a 5′-NGG protospacer-adjacent motif (PAM). In one nonlimiting example, crRNA and tracrRNA may be synthesized by in vitro transcription, using a synthetic double stranded DNA template including the T7 promoter. The tracrRNA may have a fixed sequence, whereas the target sequence may dictate part of the crRNA's sequence. Equal molarities of crRNA and tracrRNA may be mixed and heated at 55° C. for 30 seconds. Cas9 may be added at the same molarity at 37° C. and incubated for 10 minutes with the RNA mix. A 10-20 fold molar excess of the resulting Cas9-gRNA RNP then may be added to the target DNA. The binding reaction may occur within 15 minutes. Other suitable reaction conditions readily may be used.

As used herein, the term “nuclease” is intended to mean an enzyme capable of cleaving the phosphodiester bonds between the nucleotide subunits of polynucleotides. The term “endonuclease” refers to an enzyme capable of cleaving the phosphodiester bond within a polynucleotide chain; and the term “nickase” refers to an endonuclease which cleaves only a single strand of a DNA duplex. The term “Cas9 nickase” refers to a nickase derived from a Cas9 protein, typically by inactivating one nuclease domain of Cas9 protein.

In the context of a polypeptide, the terms “variant” and “derivative” as used herein refer to a polypeptide that includes an amino acid sequence of a polypeptide or a fragment of a polypeptide, which has been altered by the introduction of amino acid residue substitutions, deletions or additions. A variant or a derivative of a polypeptide can be a fusion protein which contains part of the amino acid sequence of a polypeptide. In the context of a polypeptide, the term “variant” or “derivative” as used herein also refers to a polypeptide or a fragment of a polypeptide, which has been chemically modified, e.g., by the covalent attachment of any type of molecule to the polypeptide. For example, but not by way of limitation, a polypeptide or a fragment of a polypeptide can be chemically modified, e.g., by glycosylation, acetylation, pegylation, phosphorylation, amidation, derivatization by known protecting/blocking groups, proteolytic cleavage, linkage to a cellular ligand or other protein, etc. The variants or derivatives are modified in a manner that is different from naturally occurring or starting peptide or polypeptides, either in the type or location of the molecules attached. Variants or derivatives further include deletion of one or more chemical groups which are naturally present on the peptide or polypeptide. A variant or a derivative of a polypeptide or a fragment of a polypeptide can be chemically modified by chemical modifications using techniques known to those of skill in the art, including, but not limited to specific chemical cleavage, acetylation, formulation, metabolic synthesis of tunicamycin, etc. Further, a variant or a derivative of a polypeptide or a fragment of a polypeptide can contain one or more non-classical amino acids. A polypeptide variant or derivative may possess a similar or identical function as a polypeptide or a fragment of a polypeptide described herein. A polypeptide variant or derivative may possess an additional or different function compared with a polypeptide or a fragment of a polypeptide described herein.

As used herein, the term “sequencing” is intended to mean determining the sequence of a polynucleotide. Sequencing may include one or more of sequencing-by-synthesis (SBS), bridge PCR, chain termination sequencing, sequencing by hybridization, nanopore sequencing, and sequencing by ligation.

As used herein, the term “species specific repetitive element” is intended to mean a repeating sequence that occurs within the polynucleotides of a given species and that may not occur within the polynucleotides of another species. A species having multiple chromosomes (such as mammal, e.g., human) may include different species specific elements on each chromosome, or may include the same species specific element on each chromosome, or a mixture of same and different species specific elements on each chromosome. One example of a species specific repetitive element is a photospacer adjacent motif, or PAM sequence, such as NGG. The gRNA of a Cas-gRNA RNP may have a sequence that hybridizes to a species specific repetitive element.

As used herein, the terms “unique molecular identifier” and “UMI” are intended to mean an oligonucleotide that may be coupled to a polynucleotide and via which the polynucleotide may be identified. For example, a set of different UMIs may be coupled to a plurality of different polynucleotides, and each of those polynucleotides may be identified using the particular UMI coupled to that polynucleotide.

As used herein, to be “selective” for an element is intended to mean to couple to that target and not to couple to a different element. For example, a Cas-gRNA RNP that is selective for a species specific repetitive element may couple to that species specific repetitive element and not to a different species specific repetitive element. When used in reference to a guide RNA or other polynucleotide, terms such as “target specific” and “selective” are intended to mean a polynucleotide that includes a sequence that is specific to (substantially complementary to and may hybridize to) a sequence within another polynucleotide.

As used herein, the terms “complementary” and “substantially complementary,” when used in reference to a polynucleotide, are intended to mean that the polynucleotide includes a sequence capable of selectively hybridizing to a sequence in another polynucleotide under certain conditions.

As used therein, terms such as “amplification” and “amplify” refer to the use of any suitable amplification method to generate amplicons of a polynucleotide. Polymerase chain reaction (PCR) is one nonlimiting amplification method. Other suitable amplification methods known in the art include, but are not limited to, rolling circle amplification; riboprimer amplification (e.g., as described in U.S. Pat. No. 7,413,857): ICAN: UCAN; ribospia; terminal tagging (e.g., as described in U.S. 2005/0153333); and Eberwine-type aRNA amplification or strand-displacement amplification. Additional, nonlimiting examples of amplification methods are described in WO 02/16639: WO 00/56877; AU 00/29742; U.S. Pat. No. 5,523,204; 5,536,649; 5,624,825; 5,631,147; 5,648,211; 5,733,752; 5,744,311; 5,756,702; 5,916,779; 6,238,868; 6,309,833; 6,326,173; 5,849,547; 5,874,260; 6,218,151; 5,786,183; 6,087,133; 6,214,587; 6,063,604; 6,251,639; 6,410,278; WO 00/28082; U.S. Pat. Nos. 5,591,609; 5,614,389; 5,773,733; 5,834,202; 6,448,017; 6,124,120; and 6,280,949.

The terms “polymerase chain reaction” and “PCR,” as used herein, refer to a procedure wherein small amounts of a polynucleotide, e.g., RNA and/or DNA, are amplified. Generally, amplification primers are coupled to the polynucleotide for use during the PCR. See, e.g., the following references, the entire contents of which are incorporated by reference herein: U.S. Pat. No. 4,683,195 to Mullis: Mullis et al., Cold Spring Harbor Symp. Quant. Biol., 51: 263 (1987); and Erlich, ed., PCR Technology, (Stockton Press, NY, 1989). A wide variety of enzymes and kits are available for performing PCR as known by those skilled in the art. For example, in some examples, the PCR amplification is performed using either the FAILSAFE™ PCR System or the MASTERAMP™ Extra-Long PCR System from EPICENTRE Biotechnologies, Madison, Wis., as described by the manufacturer.

As used herein, terms such as “ligation” and “ligating” are intended to mean to form a covalent bond or linkage between the termini of two or more polynucleotides. The nature of the bond or linkage may vary widely and the ligation may be carried out enzymatically or chemically. Ligations may be carried out enzymatically to form a phosphodiester linkage between a 5′ carbon terminal nucleotide of one oligonucleotide with a 3′ carbon of another nucleotide. Template driven ligation reactions are described in the following references, the entire contents of each of which are incorporated by reference herein: U.S. Pat. Nos. 4,883,750; 5,476,930; 5,593,826; and 5,871,921. Ligation also may be performed using non-enzymatic formation of phosphodiester bonds, or the formation of non-phosphodiester covalent bonds between the ends of polynucleotides, such as phosphorothioate bonds, disulfide bonds, and the like.

In the context of polynucleotides, the term “variant” is intended to mean that a given polynucleotide has a sequence that is different by at least one base than the sequence of another polynucleotide, such as an original genomic sequence.

As used herein, the term “saturationally mutagenized” is intended to mean that every base in a gene is substituted with the other three bases.

As used herein, the term “library” is intended to mean a collection or plurality of polynucleotides which share common sequences at their 5′ ends and common sequences at their 3′ ends, and which have different sequences than one another between those common sequences. As one example, a library of saturationally mutagenized polynucleotides refers to a collection of polynucleotides which share common sequences at their 5′ ends and common sequences at their 3′ ends, and in which every base in a given gene in those polynucleotides is substituted with the three other bases. As another example, a library of genomically edited polynucleotides refers to a collection of polynucleotides which share common sequences at their 5′ ends and common sequences at their 3′ ends, and in which different ones of the polynucleotides are genomically edited in different ways than one another.

Analyzing Expression of Protein-Coding Variants in Cells

Currently available variant assays can be low throughput. For example, currently available approaches to assays for variants with unknown function are limited by specific phenotypic assays. Such approaches may provide limited information about variants, and also may be difficult to scale up to many genes in a high throughput manner because each gene requires a different assay. The inventors are unaware of any work using scRNA-seq as a read out for saturationally edited variants.

In comparison, some examples herein may provide a high throughput variant assay using scRNA-seq with saturationally edited genes. These examples use scRNA-seq as a readout of genome editing that provides rich information from many genes and/or pathways on molecular, cellular, and organismal phenotypes, is generalizable to all genes, and provides significantly more fine grained information about variant function. As provided herein, scRNA-seq may be used as a read-out for variant function within a generic workflow, for any exon mutations in a gene. For example, the present inventors recognized that a challenge of using scRNA-seq for a high throughput variant assay is how to link (associate) cell barcodes to variants for a large set of variants, especially for exons far away from the transcript termini. As provided herein, a knock-in mutagenesis method may be used to link cell barcodes with edited variants, at the same time as creating the edited variant allele.

Some examples herein may introduce a barcoded saturationally mutagenized variant library into the cell, and use scRNA-seq as a read-out to assay for the variant effect. In this approach, every base in the coding region of the protein may be mutagenized to the other three alternative bases, thereby generating up to 9 different amino acids or stop codons for each codon. Therefore, the functional impact of every possible variant on the coding region of every gene can be assayed. For example, the present inventors recognized that a challenge of using scRNA-seq for a high throughput variant assay is how to link cell barcodes to variants for a large set of variants. As provided herein, a randomly barcoded vector may be used to barcode each variant on the UTR region, and read this variant barcode out in scRNA-seq. With a separate sequencing (amplicon sequencing or long read sequencing), the variant barcodes may be linked to the variants.

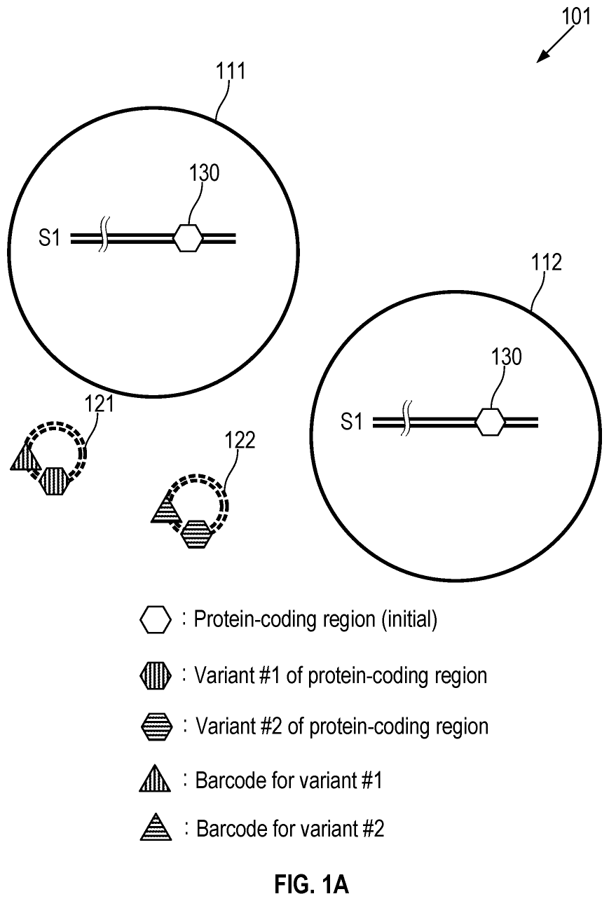

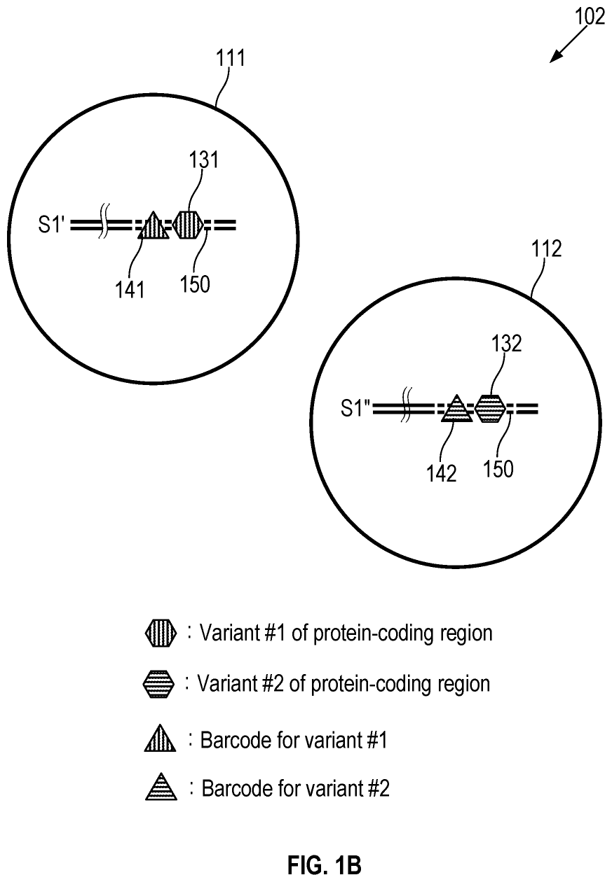

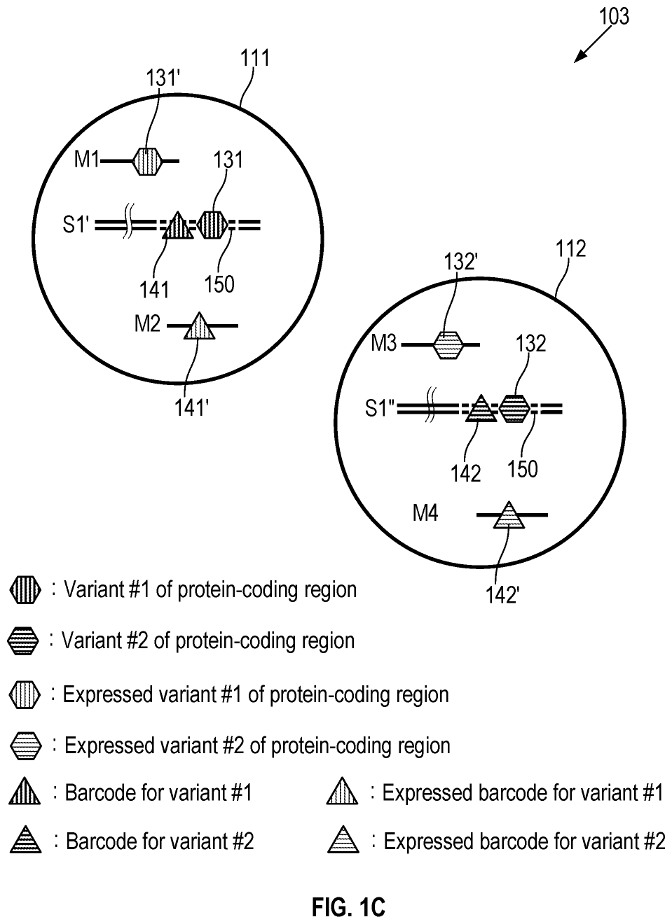

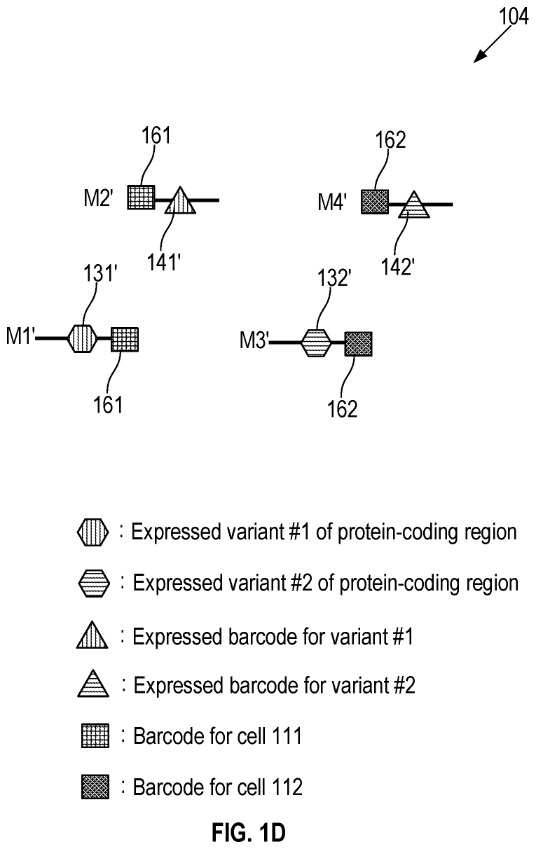

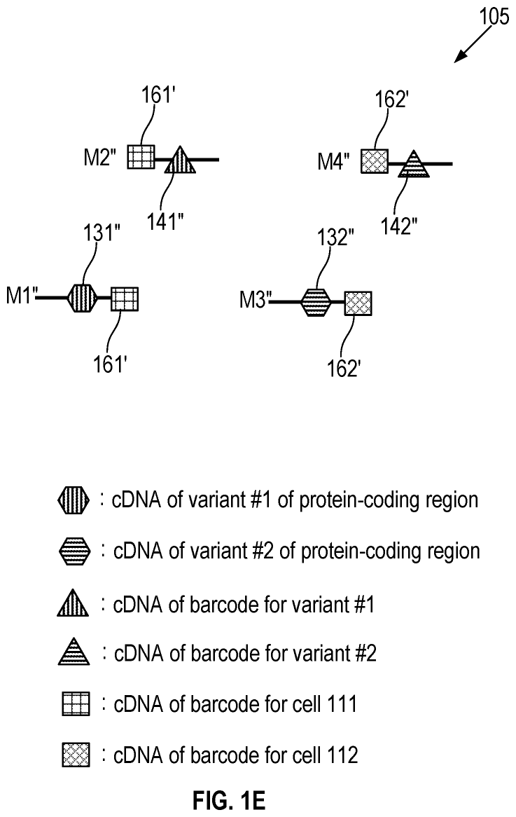

FIGS. 1A-IE schematically illustrate example compositions and operations in a process flow for analyzing expression of protein-coding variants in cells. Composition 101 illustrated in FIG. 1A includes cells 111 and 112 for which it is desired to analyze the expression of different protein-coding variants. For example, cells 111 and 112 initially may include the same DNA sequence S1 including the same protein coding region 130, illustratively a naturally occurring protein-coding region. The cells' expression of region 130 may be well characterized, and it may be desired to determine the effect, if any, of changes to the DNA sequence of that protein coding region on the cells' expression of that protein coding region. As provided herein, protein coding region 130 in cells 111, 112 may be replaced using a donor vector 121, 122 that includes a variant of the protein-coding region and a first barcode identifying that variant. Illustratively, composition 101 may include vectors 121, 122 that are brought into contact with cells 111, 112. It will be appreciated that although FIG. 1A illustrates the use of two cells and two vectors for simplicity, operations and compositions such as described with reference to FIGS. 1A-1E may be used for any suitable number of cells and for any suitable number of vectors, e.g., any suitable combination of one cell, or more than one cell, or more than ten cells, or more than 100 cells, or more than 1,000 cells, or more than 10,000 cells, or even more than 100,000 cells, and one vector, or more than one vector, or more than ten vectors, or more than 100 vectors, or more than 1,000 vectors, or more than 10,000 vectors, or even more than 100,000 vectors.

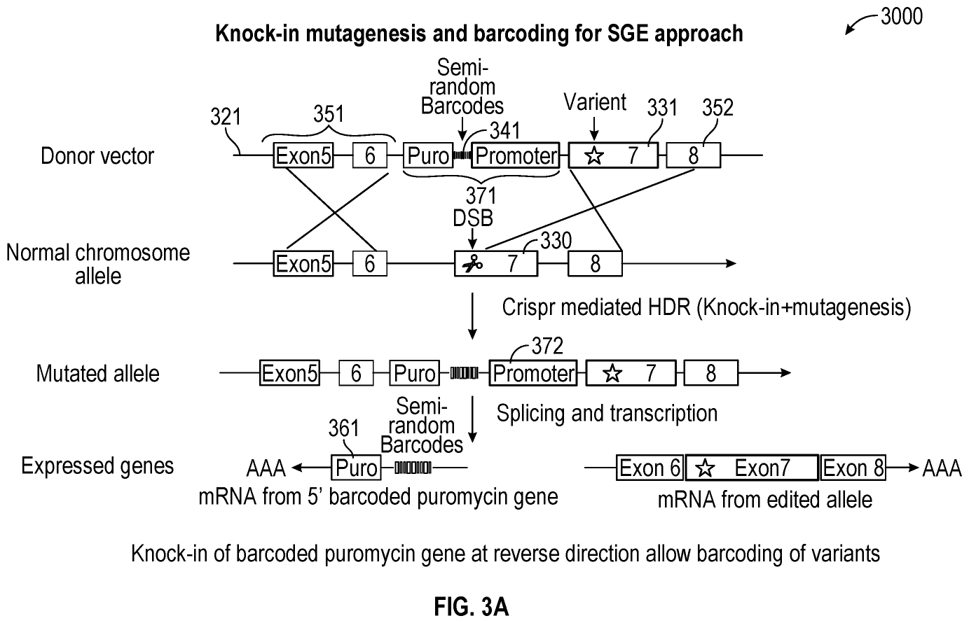

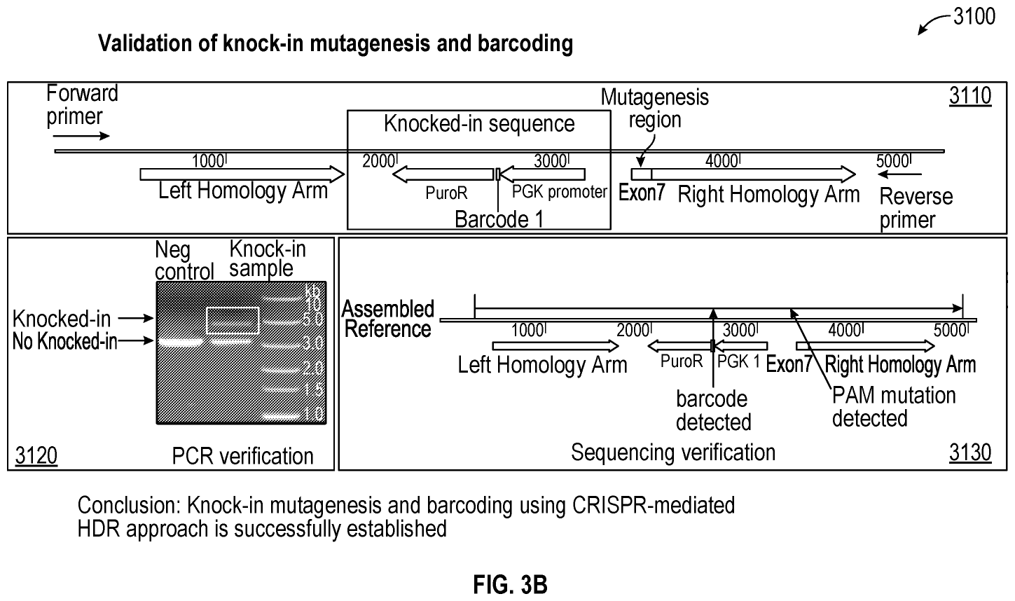

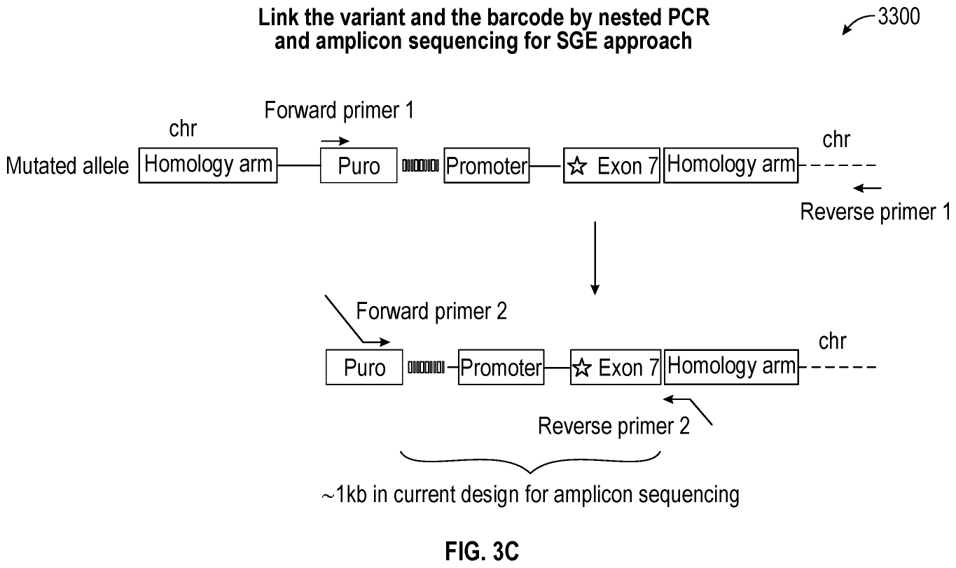

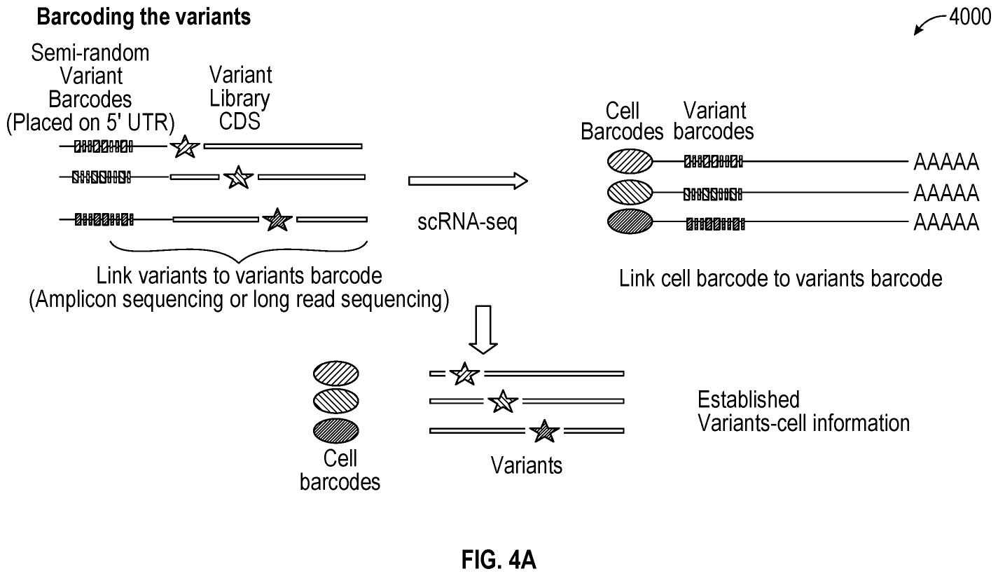

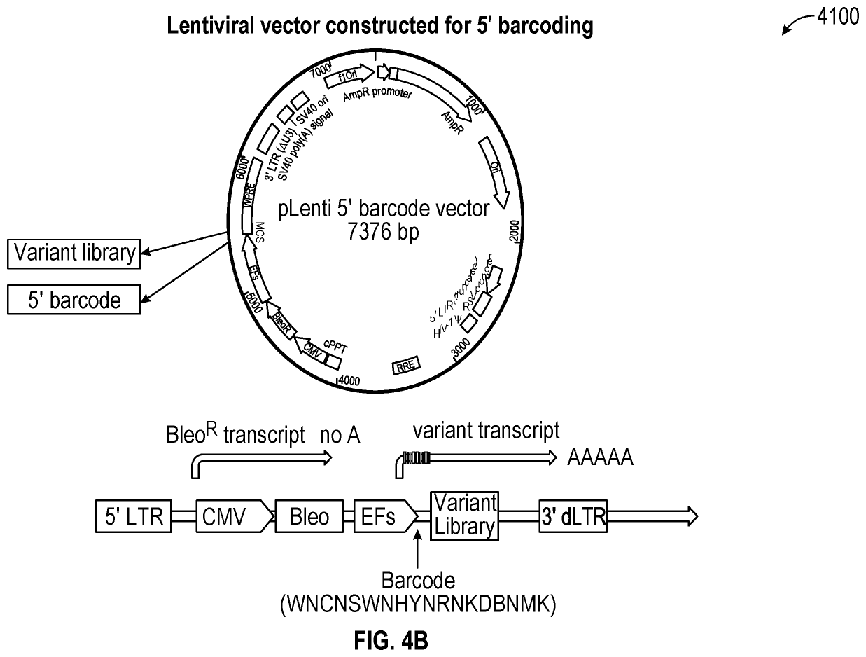

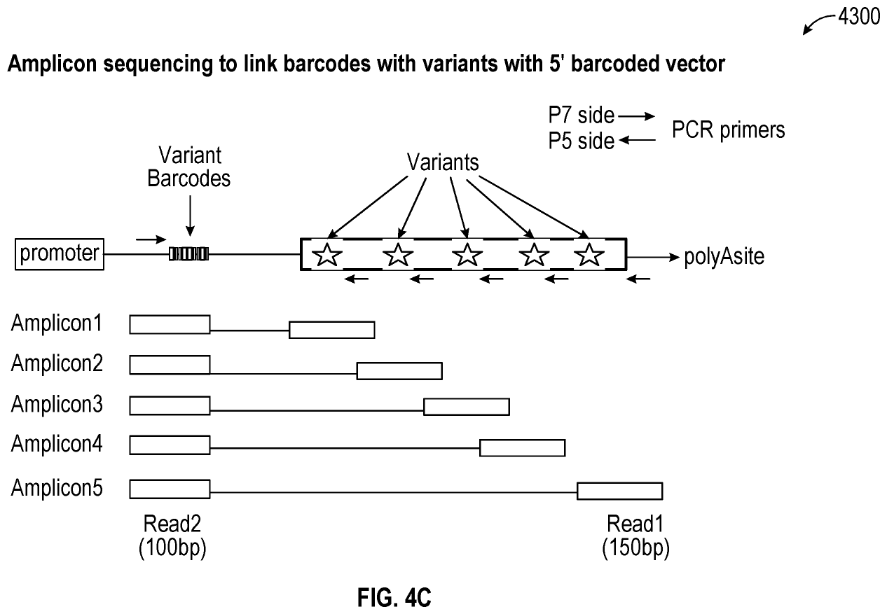

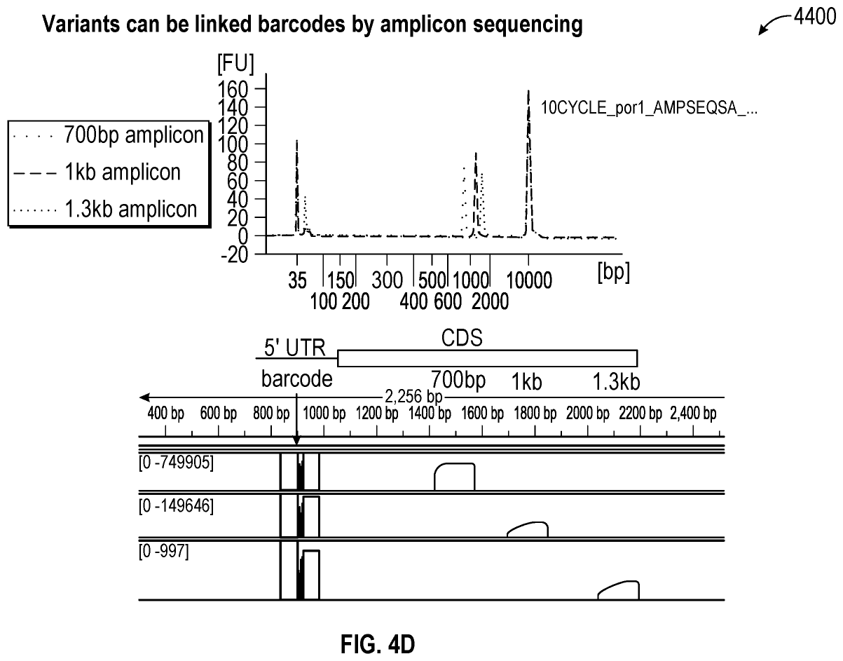

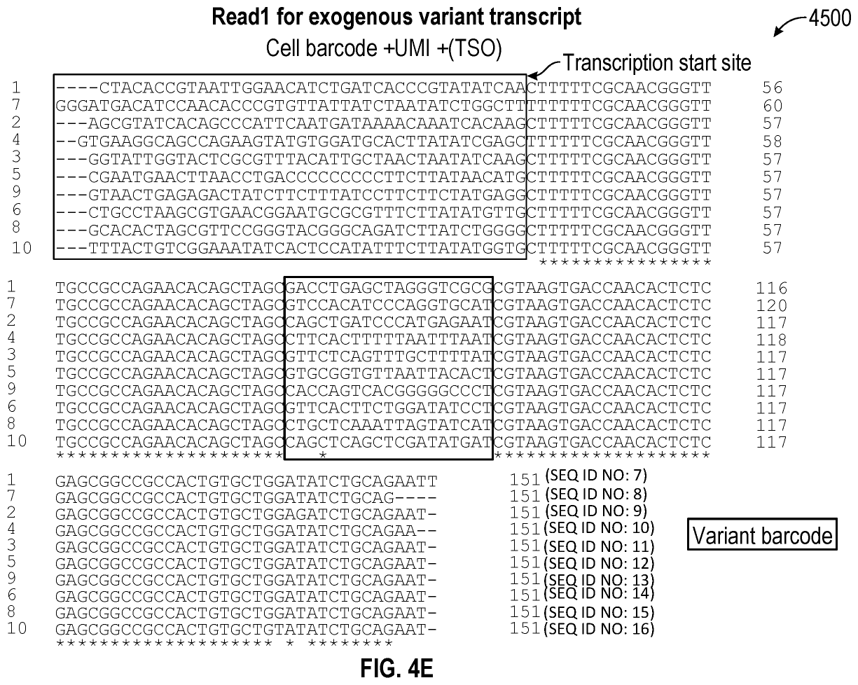

As illustrated in composition 102 of FIG. 1B, vectors 121, 122 from FIG. 1A may be used to replace protein coding region 130 from FIG. 1A in respective cells 111, 112 with a respective variant 131, 132 that includes a sequence varying from protein coding region 130 by at least one base, as illustrated in composition 102 of FIG. 1B. Additionally, vectors 121, 122 may be used to insert a respective first barcode 141, 142 into the DNA of cells 111, 112 that corresponds to the variant. Optionally, additional portions 150 of vectors (e.g., one or more additional bases on either side of variant 131, 132, on either side of first barcode 141, 142, and/or between the variant and its respective barcodes) also may be inserted into the DNA of cells 111, 112. As a result of replacing protein coding region 130 with respective variants and inserting respective barcodes into DNA sequence S1, cells 111, 112 may have different sequences than one another. For example, cell 111 may include DNA sequence S1 including variant 131 and first barcode 141, and cell 112 may include DNA sequence S1″ including variant 132 and first barcode 142. Variant 131 may have a different sequence than variant 132, and first barcode 141 may have a different sequence than first barcode 142. Nonlimiting examples of vectors and operations for replacing coding regions with respective variants coupled to barcodes are described elsewhere herein, e.g., with reference to FIGS. 3A-3C and 4A-4E.

As illustrated in composition 103 of FIG. 1C, cells 111, 112 may express DNA sequences S1′, S1″ to generate mRNA that respectively includes an expression of the variant 131, 132 and an expression of the corresponding first barcode 141, 142. Illustratively, cell 111 may express sequence S1′ as mRNA molecule M1 which includes expression 131′ of variant 131, and as mRNA molecule M2 which includes expression 141′ of first barcode 141. Similarly, cell 112 may express sequence S1″ as mRNA molecule M3 which includes expression 132′ of variant 132, and as mRNA molecule M4 which includes expression 142″ of first barcode 142. It will be appreciated that because variants 131 and 132 have different sequences than one another, cells 111 and 112 may express those sequences differently than one another. For example, differences in the sequences of variants 131 and 132 may have different effects on the respective cells' regulation of gene expression, and it may be desirable to analyze such effects and to compare such effects to one another. Such information may be used to understand the function of the variants, since some or all variants initially may have unknown function, to increase the actionability of the genome for disease diagnostics and treatment, and/or to speed up the drug discovery process.

The respective sequences of the variant and of the mRNA generated through the cell's expression of that variant, may be correlated. In some examples, the sequence of the mRNA is determined using single cell RNA sequencing (scRNA-seq). The scRNA-seq may include coupling to the mRNA a second barcode corresponding to the cell. For example, as illustrated in FIG. 1D, a barcode molecule 161 corresponding to cell 111 may be coupled to mRNA molecule M1 to form molecule M1′ including expressed variant 131′, and another barcode molecule 161 corresponding to cell 111 may be coupled to mRNA molecule M2 to form molecule M2′ including expressed first barcode 141′. Additionally, barcode molecule 162 corresponding to cell 112 may be coupled to mRNA molecule M3 to form molecule M3′ including expressed variant 132′, and another barcode molecule 162 corresponding to cell 112 may be coupled to mRNA molecule M4 to form molecule M4′ including expressed first barcode 142′. Note that barcodes 141′, 142′ are inside of the respective transcripts M2′, M4′, while barcodes 161, 162 are coupled to the termini of the respective transcripts M1′, M2′, M3′, M4′. Optionally, the barcodes may be coupled to the mRNA molecules as part of a process for releasing the mRNA from the respective cells. The mRNA molecules then may be pooled together, as in composition 104 illustrated in FIG. 1D.

The mRNA, having the second barcodes respectively coupled thereto, may be reverse transcribed into complementary cDNA, for example as another scRNA-seq operation. For example, as illustrated in FIG. 1E, mRNA molecule M1′ may be reverse transcribed into cDNA molecule M1″ including cDNA 131″ of expressed variant 131′ of FIG. 1D, and mRNA molecule M2′ may be reverse transcribed into cDNA molecule M2″ including cDNA 141″ of expressed first barcode 141′ of FIG. 1D. Similarly, mRNA molecule M3′ may be reverse transcribed into cDNA molecule M3″ including cDNA 132″ of expressed variant 132′ of FIG. 1D, and mRNA molecule M4′ may be reverse transcribed into cDNA molecule M4″ including cDNA 142″ of expressed first barcode 142′ of FIG. 1D.

The resulting cDNA then may be sequenced, for example as another scRNA-seq operation. In this regard, note that scRNA-seq operations such as described with reference to FIGS. 1D-1E may be performed using known techniques, and indeed optionally may be performed using commercially available technology, such as the CHROMIUM Single Cell 3′ Solution available from 10× Genomics (Pleasanton, California). Additionally, the donor vectors 121, 122, mutagenized library DNA S1′, S1″, and/or cDNA M1″, M2″, M3″, M4″ may be sequenced using known techniques, such as amplicon sequencing utilizing sequencing by synthesis (SBS) of amplicons to link variants 131, 132 to the respective variant barcodes 141, 142. Such amplicon sequencing may be performed in a manner such as described with reference to FIG. 4C, and the SBS optionally may be performed using commercially available technology, such as the MISEQ System available from Illumina, Inc. (San Diego, California).

The donor vector sequence and the cDNA sequence may be correlated with one another to identify the variant and the cell's expression of the variant. For example, referring to FIG. 1E, although cDNA molecules M1″, M2″, M3″, M4″ are pooled, barcodes 161′ may be correlated to determine that cDNA molecules M1″ and M2″ came from the same cell as one another, because the same barcode occurs in the sequence of both molecules. Similarly, barcodes 162′ may be correlated to determine that cDNA molecules M3″ and M4″ came from the same cell as one another, because the same barcode occurs in the sequence of both molecules. Additionally, referring to FIG. 1A, although it may not necessarily be known or controlled which particular donor vector is used to add the corresponding variant 131 or 132 into which cell, the respective sequences of the donor vectors may be correlated to determine that barcode 141 corresponds to protein-coding region 131 because they are in the same molecule as one another, and that barcode 142 corresponds to protein-coding region 132 because they are in the same molecule as one another. Accordingly, referring again to FIG. 1E, based on correlation between the scRNA-seq sequences with the donor vector sequences, it may be determined that cDNA barcode 141′ corresponds to variant 131″ and that variant 131″ was within cell 111, and that cDNA barcode 142′ corresponds to variant 132″ and that variant 132″ was within cell 112. Such correlation may be referred to as “linking” the cell barcode to the variant.

In some examples, nested polymerase chain reaction (PCR) operations may be used to sequence the donor vector, which may be relatively long. For example, in a manner such as described with reference to FIG. 3C, a first process may be used to generate a first amplicon of the donor sequence that includes the variant, the first barcode, and the right homology arm and substantially excludes the left homology arm. Then, a second PCR process may be used to generate a second amplicon of the first amplicon that includes the variant and the first barcode and substantially excludes the right and left homology arms. Sequencing the donor vector may include sequencing the second amplicon, which in some examples may have a length of about 1000 bases or fewer. Another example process for sequencing a donor vector is described with reference to FIG. 4C.

It will be appreciated that any suitable donor vectors may be used to replace protein-coding regions in cells with any suitable variants, and to add first barcodes corresponding to such variants. In some examples, the donor vector may include a promoter region, e.g., that the cell may use to initiate expression of the barcode, the variant, or both the barcode and the variant. Illustratively, the barcode may be located between the promoter region and the variant, in which case the cell may use the promoter region to initiate expression of both the barcode and the variant in a manner such as described in greater detail with reference to FIGS. 4A-4E. In other examples, the promoter region may include a reverse promotor region, and optionally the reverse promoter region is disposed between the first barcode and the variant, in which case the cell may use the reverse promoter region to initiate expression of either the barcode or the variant in a manner such as described with reference to FIGS. 3A-3C. For example, the expression of the variant of the protein-coding region may be in the forward direction, and the expression of the first barcode may be in the reverse direction, in a manner such as described with reference to FIGS. 3A-3C. Additionally, or alternatively, the donor vector may include right and left homology arms, the variant and the first barcode being between the right and left homology arms in a manner such as described with reference to FIGS. 3A-3C.

Turning now to FIGS. 3A-3C, example compositions and operations in a process for random barcoded saturation genome editing for a high throughput protein coding variant assay by single cell RNA-seq (scRNA-seq) are schematically illustrated. As illustrated in FIG. 3A, a randomly barcoded homology donor vector 321 may be constructed by putting a semi-random barcode 341 within or on the UTR termini of a foreign transcript 371 that links to a promoter and puromycin resistance gene in the illustrated example. Variant 331 of a protein coding region may be located adjacent to foreign transcript 371. On the one hand, this donor vector 321 may include homology arms 351, 352 and desired mutations on the donor repair template (e.g., within variant 331 of a protein coding region) to create variants on the exon which subsequently may be cleaved by a Cas-gRNA RNP to generate a double stranded break (DSB) within or near the protein-coding region 330 on a normal allele, and cause the cell to initiate a homology directed repair (HDR) process by which variant 331 is used to replace the normal protein-coding region 330. On the other hand, this foreign gene 371 with semi-random barcodes 341 may be knocked into the vicinity of the exon to be edited in the reverse orientation. The semi-random barcode is placed on the UTR termini of the foreign gene 371 so that it may be expressed and detectable in scRNA-seq. A non-limiting example of knock-in mutagenesis using puromycin resistance gene is illustrated in FIG. 3A. In FIG. 3A it may be seen that the foreign gene 371 may include reverse promoter 372 in the intron: as such, the foreign gene 371 driven by reverse promoter 372 can be spliced out and will not affect the normal protein translation. The cell expresses semi-random barcodes 341 driven by reverse promoter that are linked to variant 331, and expresses semi-random barcodes 341 and puromycin resistance gene 361, in the reverse direction, into a first mRNA molecule; and expresses variant 331 in the forward direction into a second mRNA molecule. For example, barcode 341 may be located on a UTR terminus of the puromycin resistance gene, and the cell later may be contacted with puromycin to enrich for the cell. Homology donor vector 231 may be inserted into the cell by inserting into the cell a plasmid on which the donor vector is located. Additionally, a second plasmid may be inserted into the cell that causes the cell to express Cas-gRNA RNP for use in the HDR process.

FIG. 3B contains preliminary data which demonstrates the barcoded puromycin resistance gene can be successfully knocked-in and the exon can also be successfully edited. The top panel 3110 of FIG. 3B illustrates the position and size of the knocked-in part, where the mutation should be. In panel 3120, the band on gel for PCR verification in the red box shows the band generated after successful knock-in mutagenesis. In panel 3130, sequencing verification shows that the barcode and variants have been successfully introduced. This shows that the present “knock-in mutagenesis” approach works and can be used to barcode the variants.

To link a barcode with edited variants, a two-step PCR and amplicon sequencing may be performed in a manner such as illustrated in FIG. 3C. The first PCR may specifically amplify the knocked-in region with genomically edited allele. The second PCR may use the PCR product from the first PCR as a template, and may link the barcode with variants in a ˜1 kb amplicon. An amplicon sequencing is performed using the product from the second PCR. Amplicon sequencing may be performed using commercially available sequencers, such as the MISEQ sequencer that is commercially available from Illumina, Inc. (San Diego, CA).

To link the cell barcode with the variant barcode, the scRNA-seq library may be sequenced, e.g., by 150 bps, to cover both the cell barcode and the variant barcode region. In this way the cell barcode may be linked to the variant barcode using the read from the foreign transcript that is knocked into the neighboring intronic region.

A computational decoding pipeline may be used to link these two datasets (amplicon sequencing and scRNA-seq) which may decode which cells are linked to which variants. Another computational pipeline and deep learning algorithm may be used to analyze the impact of each variant on gene expression in each cell based on the cell barcode-variant relationship decoded, and scRNA-seq data.

In other examples, FIGS. 4A-4E schematically illustrate example compositions and operations in a process flow for a high throughput protein coding variant assay by single cell RNA-seq (scRNA-seq) using an exogenous variant library that is saturationally mutagenized. In a manner such as illustrated in FIG. 4A and described in greater detail with reference to FIGS. 1A-1E, a computational decoding pipeline may be used to link these two datasets which will decode which cells are linked to variants. In the barcoded vector, a semi-random barcode may be placed downstream of the promoter or upstream of the terminator for the pool of variant library to be cloned in, such that this barcode will be in the UTR region of the variant transcript after the pool of variant library is cloned in. In this way, every variant may have a unique barcode expressed in the UTR region. The variants may be linked to variant barcodes using amplicon sequencing such as described above with reference to FIG. 1E, or long read sequencing. The expressed variants and expressed variant barcodes may be linked to cell barcodes using scRNA-seq in a manner such as described with reference to FIG. 1E. The variant barcodes may be linked to the expressed variant barcodes by correlating the vector sequence to the expressed sequence in a manner such as described with reference to FIG. 1E.

FIG. 4B illustrates an example vector that may be used to insert a first barcode into a cell's DNA, and to replace a protein coding region in the cell with a variant in a manner such as described with reference to FIGS. 1A-1B or 3A. Vector 4100 illustrated in FIG. 4B may include a lentiviral vector constructed for 5′barcoding, such as a pLenti 5′ barcode vector. Using molecular cloning, the variant and first barcode may be inserted into any appropriate region of the vector, for example between the WPRE and EFs sequences. Example mRNA and protein sequences that may result from a cell's expression of the vector are also illustrated in FIG. 4B.

As noted above with reference to FIG. 1E, in some examples, to link barcodes with variants, tiled PCR amplicons may be generated by using one set of primers to amplify the barcode on one side, and another set of primers to amplify the variants on the other side. Each amplicon may be used to link a segment of the variants to the barcode. Amplicon sequencing may be performed using a modified recipe on a sequencer, such as a MISEQ sequencer that is commercially available from Illumina, Inc. (San Diego, CA). In this way, the variant barcodes may be linked to the variants in a manner such as illustrated in FIG. 4C, with example data illustrated in FIG. 4D using the computation pipeline with this dataset. More specifically, FIG. 4C illustrates an amplicon assay performed on vector DNA, mutagenized library DNA, and/or cDNA, in which one PCR primer (tiled across the region) is used to amplify the variant, and the other PCR primer is used to amplify the variant barcode. In some examples, commercially available SBS may only be used to perform sequencing on regions of about 150 base pairs or fewer, tiled amplicons may be used that individually cover a respective region of about 150 base pairs or fewer, but collectively cover the entire sequence. FIG. 4D shows that the amplicon sequencing works well, with desired coverage on the barcode region and variant region for use in linking the barcode and the variant.

To link the cell barcode with the variant barcode, the scDNA-seq library may be sequenced, e.g., by about 150 base pairs, to cover both the cell barcode and variant barcode region. In this way the cell barcode may be linked to the variant barcode. Example data is illustrated in FIG. 4E. FIG. 4E shows that information for both the cell barcode and the variant barcode in the same read (Read1 of scRNA-seq).

A computational pipeline and deep learning algorithm may be developed and used to analyze the impact of each variant on gene expression in each cell based on the cell barcode-variant relationship decoded, and scRNA-seq data.

It will be appreciated that any suitable combination of process flows such as described with reference to FIGS. 1A-1E, 3A-3C, 4A-4E may be used to analyze expression of a protein-coding region of DNA in a collection of cells. For example, the initial protein coding-region of the DNA in each of the cells may be replaced with a donor vector that includes a variant of the protein-coding region and a first barcode identifying that variant, wherein the cells receive different variants than one another. mRNA may be obtained from the cells, and the mRNA from each cell may include an expression of the variant of the protein-coding region in that cell and an expression of the first barcode. The mRNA from each cell may be coupled to a second barcode corresponding to that cell. The mRNA, having the second barcode coupled thereto, may be reverse transcribed into complementary cDNA. The cDNA may be sequenced, and the donor vector also may be sequenced. The donor vector sequence and the cDNA sequence may be correlated to identify the variant in each of the cells and that cell's expression of that variant. Optionally, in some examples, such as described with reference to FIGS. 3A-3C or 4A-4E, the different variants may be saturationally mutagenized.

It will further be appreciated that as part of the present process flow, a collection of cells may be generated in which the DNA of each of the cells in the collection may include a variant of a protein-coding region and a first barcode identifying that variant. The cells may have different variants than one another. Optionally, in some examples, such as described with reference to FIGS. 3A-3C or 4A-4E, the different variants may be saturationally mutagenized.

It will further be appreciated that as part of the present process flow, a collection of polynucleotides from a collection of cells may be generated that includes first and second mRNA molecules from each of the cells. For each cell, the first mRNA molecule may include a first molecule of a barcode corresponding to that cell and an expression of a variant in that cell, and the second mRNA molecule may include the barcode corresponding to that cell and an expression of a first barcode corresponding to the variant. Optionally, in some examples, such as described with reference to FIGS. 3A-3C or 4A-4E, the different variants may be saturationally mutagenized.

It will further be appreciated that as part of the present process flow, some examples provide a plurality of lentiviral vectors, each of the lentiviral vectors including a different semi-random barcode. A mutagenically saturated variant library may be provided in contact with the plurality of lentiviral vectors.

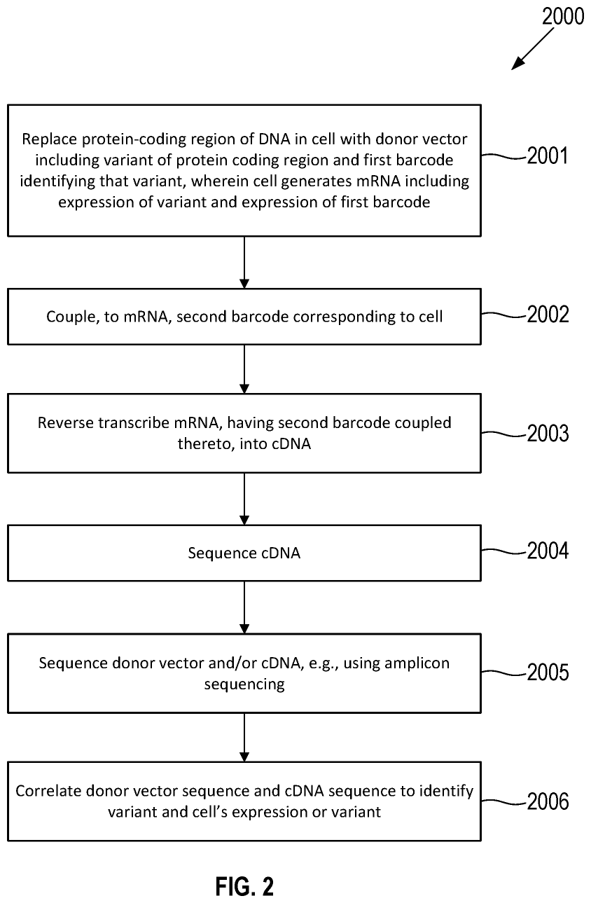

The particular vectors, compositions, and operations described herein may be modified for use in any suitable method for analyzing expression of protein-coding variants in cells. For example, FIG. 2 illustrates a flow of operations in an example method 2000 for analyzing expression of protein-coding variants in cells.

Method 2000 may include replacing a protein-coding region of the DNA in the cell with a donor vector including a variant of the protein-coding region and a first barcode identifying that variant, wherein the cell generates mRNA including an expression of the variant and an expression of the first barcode (operation 2001). For example, in a manner such as described with reference to FIGS. 1A-1B, donor vectors 121, 122 may be used to replace protein-coding region 130 of DNA sequence S1 within respective cells 111, 112 with variant 131 coupled to barcode 141 or with variant 132 coupled to barcode 142. Nonlimiting examples of vectors and of insertion methods are described with reference to FIGS. 3A and 4B. The mRNA may be generated in a manner such as described with reference to FIG. 1C.

Method 2000 also may include coupling, to the mRNA, a second barcode corresponding to the cell (operation 2002). For example, in a manner such as described with reference to FIG. 1D, the second barcode may be coupled to any mRNA molecules generated by the cell responsive to insertion of the variant and barcode. Method 2000 also may include reverse transcribing the mRNA, having the second barcode coupled thereto, into cDNA (operation 2003). For example, in a manner such as described with reference to FIG. 1E, the mRNA with second barcode may be transcribed into cDNA. Method 2000 also may include sequencing the cDNA (operation 2004). In some examples, operations 2002, 2003, 2004 are implemented in an scRNA-seq process, optionally using commercially available equipment such as described elsewhere herein. Method 2000 also may include sequencing the donor vector, and/or cDNA (operation 2005). In some examples, operation 2005 may be implemented in amplicon sequencing such as described with reference to FIG. 4C, optionally using commercially available SBS equipment such as described elsewhere herein. Optionally, the sequencing may be performed using long reads or using shortened amplicons which may be generated in a nested PCR process such as described with reference to FIG. 1E, 3C, or 4C. Method 2000 also may include correlating the donor vector sequence and the cDNA sequence to identify the variant and the cell's expression of the variant (operation 2006). Nonlimiting examples of the manner in which such correlation is performed are described with reference to FIGS. 1E, 3B, and 4A.

### WORKING EXAMPLES

The following protocols are intended to be purely illustrative, and not limiting of the present invention. In particular, it should be appreciated that the particular sizes, times, temperatures, and quantities provided are purely illustrative.

### Example 1

Nonlimiting, purely illustrative examples for Saturation Genome Editing (SGE) using CRISPR-Cas9 and Homology-directed Repair (HDR) to Study Variants of Uncertain Significance (VUS) Functions now will be described.

(A) Example Protocol of approach I: Co-transfection of sgRNA-Cas9 plasmid and barcoded variants HDR plasmid library

Introduction

Example approach I employs two sets of exon-specific plasmids to conduct saturation genome editing (SGE) in human cells. The first set of plasmids, e.g., sgRNA-Cas9 plasmids, include expression cassettes to drive the efficient expression of sgRNA and Cas9 nuclease in human cells. The sgRNAs are designed specifically for each exon of interest. The second set of plasmids, e.g., barcoded variants HDR plasmids, carry the homologous arms to the cutting site and insertion regions that include, or consist essentially of, barcoded variants and Puromycin resistance (Puro^{R}) gene. This set of plasmids are employed to induce homology-directed repair (HDR) at the cutting site while inserting the barcoded variants using Puromycin as a selection marker for later screening and enrichment. Together, these two sets of plasmids are used together to introduce a double-stranded break at a target site in human cells and subsequently carry out SGE with barcoded variants using amplicon sequencing and scRNA-Seq as readout methods.

Example Procedures

Construction of sgRNA-Cas9 plasmids. Vector backbone of sgNRA-Cas9 plasmid is linearized using PCR into two fragments (e.g., about 4-5 kb each) and subsequently purified with E-gel. sgRNAs are designed through IDT online tool, and gBlocks gene fragments including, or consisting essentially of, the sgRNAs and the overlapping regions with the backbone are ordered through IDT. Subsequently, sgRNA-Cas9) plasmids are constructed using NEBuilder HiFi DNA Assembly kit and transformed into Endura electrocompetent cells. After colonies are formed, random colonies are picked from the plate and inoculated into LB broth with Ampicillin for overnight growth. Qiagen Mini-Prep kit is then used to extract the plasmids from the cell pallet. The constructed plasmids are then subject to full-plasmid Sanger Sequencing for sequence verification.

Construction of barcoded variants HDR plasmid library. Vector backbone of HDR template plasmid is linearized using PCR (e.g., about 5.3 kb) and subsequently purified with E-gel. The homology arms are amplified from the genomic DNA of HAP1 Lig4 knock-out (KO) cell line using PCR. The Puro^{R} gene and random barcode region was amplified from a random-barcoded vector ordered from GenScript. Subsequently, the initial HDR template plasmids are constructed using NEBuilder HiFi DNA Assembly kit with these four fragments and subsequently transformed into Endura electrocompetent cells. Qiagen Maxi-Prep kit is used to extract plasmids from more than 10⁵ colonies grown on the agar plates. Nextera Flex Library is constructed and sequenced to verify the overall structures of the plasmids and amplicon sequencing targeting the random barcode region is used to ensure barcode diversity. Subsequently, the HDR template plasmid backbone is linearized using PCR into two fragments (e.g., about 4-5 kb each) and subsequently purified with E-gel. Oligo pools including oligos that each introduces a SNP to every nucleotide along the exon of interest is designed and ordered from IDT. The oligo pools are then amplified into dsDNAs using PCR. Finally, the HDR template plasmid backbones and the PCR products are assembled using NEBuilder HiFi DNA Assembly kit and subsequently transformed into Endura electrocompetent cells. Qiagen Maxi-Prep kit is used for another round of plasmid extraction from more than, e.g., about 10⁵ colonies grown on the agar plates, yielding a plasmid pool including random-barcoded variants ready for transfection.

Transfection and enrichment of cell population with successful genome editing. The constructed sgRNA-Cas9 plasmid and barcoded variants HDR plasmid library are co-transfected into a cell line, e.g., HAP1 Lig4 KO cell line using Lipofectamine 3000 following the user guide. Briefly, cells (e.g., about 5×10⁵ cells) are seeded in each well of a multi-well (e.g., about 6-well) plate about one day prior to transfection. The cells are grown overnight to reach about, e.g., about 75% confluency. On the day of transfection, Lipofectamine 3000 Reagent (e.g., about 3.75 μL) is diluted in e.g., about 125 μL Opti-MEM Medium; e.g., about 2.5 μg total of sgRNA-Cas9 plasmid and barcoded variants HDR plasmid library (e.g., about 1.25 μg each) are also diluted in e.g., about 125 μL Opti-MEM Medium along with 5 μL P3000 Reagent. The diluted components are then combined and added into each well of the multi-well plate. After about 2 days of incubation, cells are trypsin-treated and transferred into cell-culturing flasks with e.g., about 10-mL of fresh medium. Puromycin is added to each flask to reach a final concentration of, e.g., about 1 μg/mL. The culture is split again about 5 days and about 7 days post transfection with a constant Puro selection. On day 7, e.g., about 2-mL of the culture is used to extract lysate using the Lucigen QuickExtract DNA extraction solution. The lysate is then used as the DNA template for PCRs to verify the knock-in regions.

Amplicon Sequencing to link barcodes and variants. One of the lysate PCRs on day 7 (after transfection) yields an amplicon (e.g., about 3 kb) covering the barcode, variant, and right homology arm regions that is used as the DNA template for a second round of PCR to amplify a region (e.g., about 1-kb region) covering the barcode and variant regions. Adapters and sequencing indexes are added onto the amplicons through PCRs. The amplicons are sequenced using MiSeq for 151 bases for both read 1 and read 2: both indexes are 10 bases each. The sequencing data are then analyzed using a suitable bioinformatics pipeline to establish correlation between variant barcodes and variants.

10× Genomics scRNA-Seq to study the phenotypes of the variants. On the same day of lysate extraction and amplicon sequencing (e.g., about 7 days after transfection), the cells also may be used to conduct 10× Genomics scRNA-Seq to characterize the transcriptome of single cells to study the variants. The cells are prepared following the cell preparation protocol. Briefly, e.g., about 10⁷ cells are used for each sample followed by washing with 1×PBS with, e.g., about 0.04% BSA. The washed cells are filtered through a cell strainer to remove cell debris and large clumps and resuspended to a concentration of, e.g., about 10⁶ cells/mL. After the cell preparation, the 10× Genomics scRNA-Seq is initiated by following the user guide of Chromium Next GEM Single Cell 5′ Reagent Kits v2 (Dual Index). About, e.g., 10,000 cells are used as input for GEM generation and barcoding. After post GEM RT cleanup and cDNA amplification, the 5′ gene expression (GEX) library is constructed. The library is then sequenced on the NovaSeq using an SP flowcell for 210 cycles for read one and 90 cycles for read two with 10×10 indexed reads. The generated sequencing data are analyzed using a suitable bioinformatics pipeline.

(B) Example Protocol of approach II: Co-transfection of barcoded variants linear HDR library and ribonucleoprotein (RNP)

Introduction

Example approach II utilizes barcoded variants linear HDR library (e.g., about 3 kb dsDNA) and RNP to conduct SGE. The linear HDR library is amplified using PCR from the constructed barcoded variants HDR plasmid library from approach I, including the homology arms to the cutting site and insertion regions that include, or consist essentially of, barcoded variants and Puro^{R} gene. The RNP complex is formed using purified Cas9 nuclease and sgRNA in vitro. The linear HDR library and the RNP complex are then electroporated into a suitable cell line, e.g., the HAP1 Lig4 KO cell line, to conduct SGE followed by amplicon sequencing and scRNA-Seq as the readout methods.

Example Procedures

1. Construction of barcoded variants linear HDR library. The barcoded variants HDR plasmid library constructed from approach I is used as the DNA template for PCR to generate the linear HDR library, including, or consisting essentially of, the homology arms to the cutting site, random barcode, Puro^{R} gene, and variant regions. The PCR product is purified and concentrated using Zymo DNA Clean & Concentrator kit following the user guide.

2. RNP complex formation. Alt-R CRISPR-Cas9 sgRNA and Alt-R S.p. HiFi Cas9 Nuclease V3 are purchased from IDT. To form the RNP complex, 5.3 μL sgRNA (100 μM stock solution), 7.3 μL Cas9 nuclease (62 μM stock solution), and 9.4 μL DPBS are mixed per reaction in a 0.5-mL centrifuge tube and incubated at room temperature for 20 min for RNP complex formation.

3. Cell preparation and electroporation. The following protocol is modified from the electroporation of RNP user guide from IDT. Briefly, the cell culture medium is refreshed about 1 day before electroporation. On the day of electroporation, trypsin cells are placed into a flask (e.g., about 30-mL flask), then add medium to, e.g., about 10 mL and quantify the cells. Dilute, e.g., about 1×10⁷ cells into, e.g., about 40 mL by DPBS (for about 10 reactions), and centrifuge at, e.g., about 200×g for, e.g., about 5 min at room temperature. Remove supernatant without disturbing the pellet, and wash cells in 5 mL DPBS. Centrifuge at, e.g., about 200×g for about 5 min at room temperature. Remove supernatant and resuspend the cells in, e.g., about 600 μL DPBS, resulting in, e.g., about 1×10⁶ cells per 60 μL. Aliquot, e.g., about 60 μL of the resuspended cells for each electroporation in, e.g., about 1.5 mL microcentrifuge tubes. Keep the cells on ice for at least about 5 min before electroporation.

For electroporation, prepare a multi-well plate (e.g., about 6-well plate) filled with about, e.g., 2 mL of culture media per well in an approximately 37C incubator. Mix the following ingredients in, e.g., about a 0.5-mL centrifuge tube: about 20 μL of Alt-R RNP complex from step 2, about 5 μL of Alt-R electroporation enhancer (about 96 μM), about 15 μL of double-stranded linear HDR templates from step 1 (e.g., about 100 ng/μL stock), and about 60 μL of aliquoted cell suspension. Immediately transfer the mixture to cooled cuvettes (0.2 cm gap Bio-Rad #1652082), and perform electroporation at about 150V, 2 ms pulse width, 1 pulse, unipolar polarity. After electroporation, transfer the cells to the multi-well plate (e.g., use the 20 μL pipette tips to withdraw all the cells from the cuvettes). After about 2 days of incubation, cells are trypsin-treated and transferred into cell-culturing flasks with, e.g., about 10-mL of fresh medium. Puromycin is added to each flask to reach a final concentration of, e.g., about 1 μg/mL. The culture is split again, e.g., about 5 days and 7 days post transfection with a constant Puro selection. On day 7, about 2-mL of the culture is used to extract lysate using the Lucigen QuickExtract DNA extraction solution. The lysate is then used as the DNA template for PCRs to verify the knock-in regions.

4. Amplicon Sequencing to link barcodes and variants. One of the lysate PCRs on about day 7 (after transfection) yields an amplicon (e.g., about 3 kb) covering the barcode, variant, and right homology arm regions that is used as the DNA template for a second round of PCR to amplify an approximately 1-kb region just covering the barcode and variant regions. Adapters and sequencing indexes are added onto the amplicons through PCRs. The amplicons are sequenced using MiSeq for about 151 bases for both read 1 and read 2: both indexes are about, e.g., 10 bases each. The sequencing data are then analyzed using a suitable bioinformatics pipeline to establish correlation between variant barcodes and variants.

5. 10× Genomics scRNA-Seq to study the phenotypes of the variants. On the same day of lysate extraction and amplicon sequencing (e.g., about 7 days after transfection), the cells are also used to conduct 10× Genomics scRNA-Seq to characterize the transcriptome of single cells to study the variants. The cells are prepared following the cell preparation protocol. Briefly, about, e.g., 10⁷ cells are used for each sample followed by washing with 1×PBS with 0.04% BSA. The washed cells are filtered through a cell strainer to remove cell debris and large clumps and resuspended to a concentration of, e.g., about 10⁶ cells/mL. After the cell preparation, the 10× Genomics scRNA-Seq is initiated by following the user guide of Chromium Next GEM Single Cell 5′ Reagent Kits v2 (Dual Index). About, e.g., 10,000 cells are used as input for GEM generation and barcoding. After post GEM RT cleanup and cDNA amplification, the 5′ gene expression (GEX) library is constructed. The library is then sequenced on the NovaSeq using an SP flowcell for 210 cycles for read one and 90 cycles for read two with 10×10 indexed reads. The generated sequencing data are analyzed using a suitable bioinformatics pipeline.

### Example 1 Results

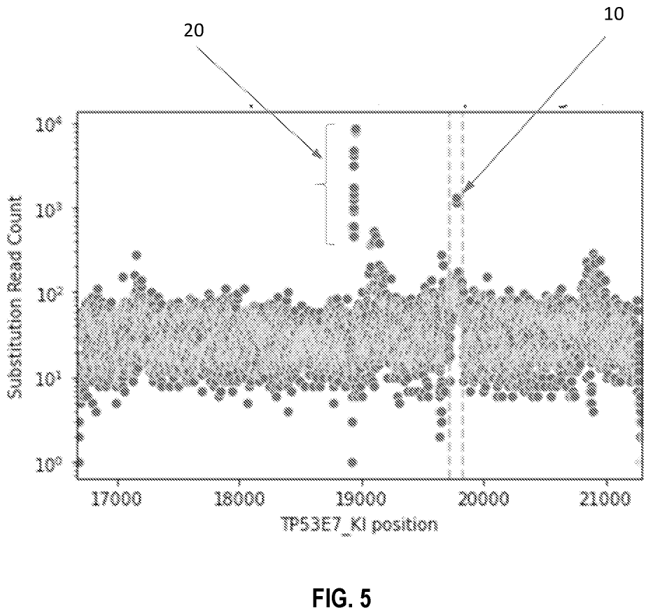

FIGS. 3B and 5 show results from a CRISPR-HDR based approach for saturation genome editing (SGE) experiment in which exon 7 of the TP53 is targeted. Example 1 provides illustrative examples of methods of SGE using CRISPR-Cas9 and Homology-directed Repair (HDR): it will be appreciated that other suitable methods may be used.

Panel 3110 of FIG. 3B illustrates a gene that contains a knocked-in sequence (puromycin gene with a barcode and mutant)). Panel 3130 of FIG. 3B also shows where the primers bind. The primers were designed to bind to sequences outside of the homology arm of the chromosome.

Cells were transfected with a vector containing the knocked-in sequence. Un-transfected cells were used as controls. PCR-generated amplicons were generated from the transfected cells and un-transfected cells, using the primers illustrated in panel 3130 of FIG. 3B.

Panel 3120 of FIG. 3B shows an agarose gel in which PCR-generated amplicons from the experimental and control cells of Example 1 were resolved. As shown in Panel 3120 of FIG. 3B, PCR-generated amplicons from the transfected cells resulted in a band that is about 1.7 kb larger than the native chromosome (˜3 kb), which is the expected size of the puromycin gene that contains the barcode and the mutant. In the un-transfected control sample, this 1.7 kb large band was absent.

FIG. 5 shows next generation sequencing of amplicons that were PCR-amplified from a saturation genome editing experiment that targeted exon 7 of TP53. In the region of the knocked-in sequence, the variant barcode and the protospacer adjacent motif (PAM) were consistently present in each of the edited genomes. Element 10 in FIG. 5 shows the location of PAM. The PAM site prevents re-cutting of the edited DNA by single guide RNA. Element 20 of FIG. 5 shows examples of variant barcodes. Together these data from FIGS. 3B and 5 show that substantially all of the bases on exon 7 of TP53 can be edited and identified using amplicon sequencing data and scRNA-seq data.

### Example 2

A nonlimiting, purely illustrative example of Cloning of library DNA into 5′UTR barcoded lentiviral vector now will be provided (Part I).

1. XhoI/BamH1 Digestion of Vector and Twist Synthesized Library

| Component | Example Amount |
|---|---|
| 5′UTR Barcoded lentiviral vector PL2 | Up to 1 ug |
| or twist synthesized library | |
| 10X NEB buffer 3.1 (in NEB R0136S) | 5 ul |
| XhoI (NEB R0146S) | 1.5 ul |
| BamH1 (NEB R0136S) | 1.5 ul |
| Nuclease-free water | Add water to 50 ul total |

Seal PCR tubes and perform digestion in a thermal cycler at, e.g., about 37° C. for about 90 min.

2. Gel Extraction of Digested Product

a. Run digestion reaction on 1% E-Gel® EX Agarose Gels (ThermoFisher G402001) according to manufacturer's instruction.

b. Open the cassette and excise the desired band.

c. Use Zymoclean Gel DNA Recovery Kit (Zymo D4002) to purify the gel piece containing the desired digested DNA. Follow the manufacture's protocol to extract the DNA. Gel piece from up to four lanes can be combined into a single extraction. Elute the DNA in, e.g., about 10-20 ul.

d. Use Qubit to quantify the DNA.

3. Ligation

Use, e.g., about 20 ng of digested vector DNA and appropriate amount of digested twist library for ligation (insert: vector=about 7:1 molar ratio). Use http://nebiocalculator.neb.com/#!/ligation to calculate.

Set up the following, e.g., about 20 ul ligation reaction.

| Component | Example Amount | |
|---|---|---|
| T4 DNA Ligase Buffer (10X) | 2 | μl |
| (NEB M0202S) | | |
| Vector DNA | 20 | ng |
| Insert DNA | 7 times molar amount of vector DNA | |
| T4 DNA Ligase (NEB M0202S) | 1 | μl |

| | |
|---|---|
| Nuclease-free water | Add water to 20 ul total |

Gently mix the reaction by pipetting up and down and spin briefly.

Seal PCR tubes and perform ligation in a thermal cycler using the following program:
About room Temp about 90 min
About 65° ° C. about 10 min
About 4° C. hold
Chill on ice before transformation or store in about −20° ° C.

4. E. coli Transformation

Example competent cell to use: Lucigen Endura ElectroCompetent cell (Lucigen 60242-2)

Follow manufacture's instruction. For each transformation reaction, use, e.g., about 1 ul ligation reaction.

Spread, e.g., about 500 ul of transformants into each of, e.g., about 15 cm LB-Ampicillin agar plate (Teknova: L5004) for DNA extraction.

Also plate, e.g., about 1-2 ul of transformants (add into, e.g., about 100 ul media) into a separate, e.g., about 10 cm plate (Teknova: L1004) to count colonies and pick single colony for sanger sequencing

Do enough transformations to reach total colony number of, e.g., about >100,000 colonies for DNA extraction.

5. DNA Extraction

Extract DNA directly from the, e.g., about 15 cm plates with transformants on it. Extract enough plates to reach total colony number of, e.g., about >100,000 colonies.

a. Collect all the cells from agar plates.

b. Pipette, e.g., about 5 mL of fresh LB broth, and place, e.g., about 5-10 Rattler Plating Beads (Zymo: S1001-5) to plates and shake them slowly using the orbital shaker (5-10 mins).

c. After, e.g., about 5-10 mins, immediately collect the cells to, e.g., about 50 mL tubes, wash the plates using LB broth a few times to collect substantially all the cells.

d. Extract the DNA following manufacture's protocol from Qiagen Maxi kit (Qiagen 12162). Detailed as following

i) Centrifuge at, e.g., about 6000 g for about 15 mins at about 4C.

ii) Decant all the supernatants and resuspend the pellet (e.g., about 300-500 mg for each extraction) in, e.g., about 10 mL Buffer.

iii) Add, e.g., about 10 ml Buffer P2, mix thoroughly by vigorously inverting about 4-6 times and incubate at about room temperature (e.g., about 15-25° C.) for about 5 min.

iv) Add, e.g., about 10 ml prechilled Buffer P3, mix thoroughly by vigorously inverting about 4-6 times. Incubate on ice for about 20 min.

v) Centrifuge at, e.g., about ≥20,000×g for about 30 min at about 4° C.

vi) Equilibrate a QIAGEN-tip 500 by applying, e.g., about 10 ml Buffer QBT and allow column to empty by gravity flow.

vii) Apply the supernatant from step v) to the QIAGEN-tip and allow it to enter the resin by gravity flow.

viii) Wash the QIAGEN-tip with, e.g., about 2×30 ml Buffer QC. Allow Buffer QC to move through the QIAGEN-tip by gravity flow.

ix) Elute DNA with, e.g., about 15 ml Buffer QF into a clean 50 ml vessel.

x) Precipitate DNA by adding, e.g., about 10.5 ml (about 0.7 volumes) RT isopropanol to the eluted DNA and mix. Centrifuge at, e.g., about ≥15,000×g for about 30 min at about 4° C. Carefully decant the supernatant.

xi) Wash the DNA pellet with, e.g., about 5 ml RT 70% ethanol and centrifuge at, e.g., about ≥15,000×g for about 10 min. Carefully decant supernatant.

xii) Air-dry pellet for, e.g., about 5-10 min and redissolve DNA in, e.g., about 150 ul of TE buffer.

6. QC by Sanger Sequencing (Optional)

Pick one or more colonies (e.g., about 16 colonies) for sanger sequencing using primer 4997F EFs (example sequence tgatgtcgtgtactggctc (SEQ ID NO: 17)). This primer is expected to read the barcode region and about 00nt into the cloned gene with good quality. Additional gene specific sequencing primer may be used if the gene is, e.g., about >500 bp long.

7. QC by Whole Genome Sequencing

Prepare Nextera DNA prep library using extracted DNA, and sequence on Miseq for 2*200 bp. Check alignment to the genome and also using overlapping regions to identify variants (a suitable data analysis pipeline may be used for this).

A nonlimiting, purely illustrative example of Lentiviral packaging and titering (Part II) now will be provided.

1. Lentiviral Packaging

Example cell line to use: 293FT cell line (ThermoFisher R70007)

Example packaging plasmid to use: ViraPower™ Lentiviral Packaging Mix (ThermoFisher K497500)

Transfection can be performed with the Lipofectamine 3000 reagent (ThermoFisher Scientific, Waltham, MA) using standard protocols (See, for example, FIG. 2 of the following protocol: https://www.thermofisher.com/content/dam/LifeTech/global/life-sciences/CellCultureandTransfection/pdfs/Lipofectamine3000-Lenti Virus-AppNote-Global-FHR.pdf, the entire contents of which are incorporated by reference herein.). For best results, use a 10 cm plate column for lentiviral packaging, and only collect the viral supernatant once.

2. Concentrating Virus with PEG-it (Optional)

a) After collecting viral supernatant, Transfer supernatant to a sterile vessel and add, e.g., about 1 volume of cold PEG-it Virus Precipitation Solution (System Bioscience LV810A-1) to about every 4 volumes of Lentivector-containing supernatant. (Example: 3 ml PEG-it with 12 ml viral supernatant). Refrigerate about 3 days at about 4° C.

b) Centrifuge supernatant/PEG-it mixture at, e.g., about 1500×g for about 30 minutes at about 4° C. After centrifugation, the Lentivector particles may appear as a beige or white pellet at the bottom of the vessel.

c) Transfer supernatant to a fresh tube. Spin down residual PEG-it solution by centrifugation at, e.g., about 1500×g for about 5 minutes. Remove substantially all traces of fluid by aspiration, taking great care not to disturb the precipitated Lentiviral particles in pellet.

d) Resuspend/combine lentiviral pellets in, e.g., about 1/100 to 1/200 of original volume using cold, sterile Phosphate Buffered Saline (PBS).

3. Lentiviral Titering by Counting Zeocin Resistant Colonies

To determine the titer of lentiviral stocks, perform the following steps: (1) prepare serial dilutions of the lentiviral stocks; (2) transduce the dilutions of the lentivirus into a mammalian cell line; (3) use a standard method to select for stably transduced cells; and (4) count the colonies of the stably transduced cells (see, for example, pages 15 to Page 21 of the following protocol: https://www.thermofisher.com/document-connect/document-connect.html?url=https %3A %2F %2Fassets.thermofisher.com %2FTFS-Assets %2FLSG %2Fmanuals %2Fvirapower_lentiviral_system_man.pdf&title=VmlyYVBvd2 VyIExlbnRpdmlyYWwgRXhwcmVzc2lvbiBTeXN0ZW0=, the entire contents of which are incorporated by reference herein).

Titering is done using cell line of choice for 10× experiment. Illustratively, use 250 μg/mL Zeocin (ThermoFisher R25001) for selection in HEK293 cell and A549 cell line (for other cell lines, a kill curve may be conducted to determine appropriate amount of Zeocin to use). Count colonies on about Day 14 after crystal violet staining.

A nonlimiting, purely illustrative example for Lentiviral transduction and 10× (Part III) now will be provided.

1) Day 1 afternoon: Seed about 3 Million ATCC HEK293 cells (ATCC CRL-1573) to each about 10 cm Plate to reach about 4 million cells the next day. Seed about 3 plates, one for lentiviral transduction, one for untransduced control, and the third one to be used to count cells next day.

2) Day 2: On the day of transduction, count the number of cells using the extra plate #3. This will be used to calculate how much virus to add. Thaw the lentiviral stock and dilute the appropriate amount of virus into fresh complete medium (e.g., about 10 mL) to obtain a MOI of about 0.05. Do not vortex. Add, e.g., about 10 uL of about 6 mg/ml Polybrene (final concentration=about 6 μg/ml). Also do a plate of untransduced control.

3) Incubate at about 37° C. overnight in a humidified about 5% CO2 incubator.

4) Day 3: Replace with about 10 ml media

5) Day4: Remove the medium and wash the cells once with PBS, trypsin the cells with, e.g., about 0.25% (w/v) Trypsin-about 0.53 mM EDTA solution. Move the entire samples from about 10 cm plates to about 15 cm plates, add, e.g., about 250 μg/mL Zeocin for selection.

6) Replace the media with fresh antibiotic about every 3-4 days.

7) Watch when the untransduced control die completely.

8) After cells on untransduced cell plate die completely, cells may be harvested for 10× library prep.

9) Prepare 10× library using 10× Chromium Next Gem Single Cell 5′ reagent kit V2 targeting, e.g., about 10,000 cells, following manufacture's protocol (10× Genomics, Pleasanton, CA) (https://assets.ctfassets.net/an68im79xiti/40B71TeTOkDoIHhfq9dPxd/05ce9121d027715321d 2a9765ble9b70/CG000331_ChromiumNextGEMSingleCell5_v2_UserGuide_RevA.pdf, the entire contents of which are incorporated by reference herein).

A nonlimiting example of Amplicon sequencing to link variant barcode to variants (Part IV) now will be provided.

This part can use either cloned plasmid DNA or amplified cDNA from 10× kit as substrate for PCR to link variant barcode with variant. The PCR cycle for these two inputs are different.

The forward PCR primer on the barcode side uses staggered primer mix and has the following example sequences:

| Name | Sequence |
|---|---|
| PL2_5BC_0N_ | GTCTCGTGGGCTCGGAGATGTGTATAAGAGACAGccgccagaacac |
| B15 | agctag (SEQ ID NO: 1) |
| PL2_5BC_1N_ | GTCTCGTGGGCTCGGAGATGTGTATAAGAGACAGNccgccagaac |
| B15 | acagctag (SEQ ID NO: 2) |
| PL2_5BC_2N_ | GTCTCGTGGGCTCGGAGATGTGTATAAGAGACAGNNccgccagaa |
| B15 | cacagctag (SEQ ID NO: 3) |
| PL2_5BC_3N_ | GTCTCGTGGGCTCGGAGATGTGTATAAGAGACAGNNNccgccag |
| B15 | aacacagctag (SEQ ID NO: 4) |

Reverse primer covering the whole gene has the following example sequence in which the gene specific sequence is after the stop codon:

(SEQ ID NO: 5)
TCGTCGGCAGCGTCAGATGTGTATAAGAGACAGccagaggttgattgtc
gaca.

Other reverse primer to tile the ORF region may be designed for each gene, the example A14-ME adaptor sequence of

(SEQ ID NO: 6)
TCGTCGGCAGCGTCAGATGTGTATAAGAGACAG

may be added in front of gene specific sequence. Design primer every 100-150 bp to tile the whole gene.

Example Procedure:

1. Gene Specific PCR:

Set up the following reaction:

| Component | Example Amount | |
|---|---|---|
| DNA | 5 | ng |
| Forward primer mix (about 0.25 uM | 5 | ul |
| each oligo) | | |
| Reverse primer (Gene specific primer | 5 | ul |
| with A14-ME adaptor) about 1 uM | | |
| 2x KAPA HiFi HotStart Ready Mix | 12.5 | ul |
| (Kapa KK2602) | | |

| | |
|---|---|
| Nuclease free water | Add water to total of 25 ul volume |

Run the following PCR program
about 95° ° C. about 3 min
about 10 (for cloned plasmid DNA) or about 16 (for 10× cDNA) cycles of
   about 95ºC for about 30 seconds
   about 55° ° C., or about 60° C., for about 30 seconds
   about 72ºC for about 30 seconds
about 72° ° C. about 5 min
Hold at about 4° C.

2. Gene Specific PCR Clean Up:

a. Vortex the AMPure XP beads for about 30 seconds to make sure that the beads are evenly dispersed.

b. Add about 20 μl of AMPure XP beads (about 0.8×) to each well, gently pipette entire volume up and down about 10 times.

c. Incubate at room temperature without shaking for about 5 minutes.

d. Place on the magnetic stand and wait until the liquid is clear (about 2 minutes). Remove and discard all supernatant.

e. Wash beads with, e.g., about 200 μl fresh 80% ethanol. Remove and discard all supernatant.

f. Centrifuge briefly and Use a P20 multichannel pipette with fine pipette tips to remove excess ethanol. Allow the beads to air-dry for about 10 minutes.

g. Add, e.g., about 52.5 μl of 10 mM Tris pH 8.5 to the beads.

h. Gently pipette entire volume up and down about 10 times. Incubate at room temperature for about 2 minutes. Place on the magnetic stand and wait until the liquid is clear (about 2 minutes).

i. Carefully transfer, e.g., about 50 μl of the supernatant to a new PCR tubes and label them accordingly. 3. Index PCR

Set up the following reaction:

| Component | Example amount |
|---|---|
| Resuspended PCR product DNA | 5 ul |
| from Step2i | |
| Nextera Flex Dual Index adapters | 10 ul |
| (Illumina: IDT for Illumina | |
| DNA/RNA UD indexes: 20027213) | |
| 2x KAPA HiFi HotStart Ready Mix | 25 ul |
| (Kapa KK2602) | |
| Nuclease free water | Add water to total of 25 ul volume |

Run the following PCR program
about 95° C. about 3 min
about 8 (for cloned plasmid DNA) or about 9 (for 10× cDNA) cycles of
   about 95ºC for about 30 seconds
   about 55° ° C., or about 60° C., for about 30 seconds
   about 72ºC for about 30 seconds
about 72° ° C. about 5 min
Hold at 4° C. 4. Index PCR Clean Up

a Vortex the AMPure XP beads for about 30 seconds to make sure that the beads are evenly dispersed.

b. Add, e.g., about 50 μl of AMPure XP beads (1×) to each well, gently pipette entire volume up and down about 10 times.

c. Incubate at room temperature without shaking for about 5 minutes.

d. Place on the magnetic stand and wait until the liquid is clear (about 2 minutes). Remove and discard substantially all supernatant.

e. Wash beads with, e.g., about 200 μl fresh 80% ethanol. Remove and discard all supernatant.

f. Centrifuge briefly and Use a P20 multichannel pipette with fine pipette tips to remove excess ethanol. Allow the beads to air-dry for about 10 minutes.

g. Add, e.g., about 27.5 ul of 10 mM Tris pH 8.5 to the beads.

h. Gently pipette entire volume up and down about 10 times. Incubate at room temperature for about 2 minutes, Place on the magnetic stand and wait until the liquid is clear (about 2 minutes).

i. Carefully transfer, e.g., about 25 μl of the supernatant to a new PCR tubes and label them accordingly.

5. Quantitate Library

Run, e.g., about 1 μl of an about 1:20 dilution of the final library on a Bioanalyzer DNA High Sensitivity Chip to get final concentration of the library. Expect to see a single peak for each PCR. Choose the peak to quantitate.

6. Sequencing

Mix library with at least about 5% phiX (FC-110-3001) to sequence on Miseq or Novaseq.

### Example 2 Results

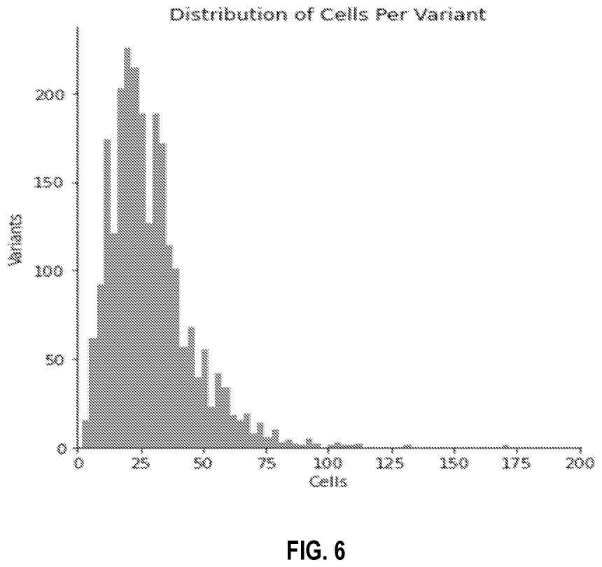

FIG. 6 illustrates a distribution of variants of a saturationally mutagenized TP53 library which was introduced into HEK293 cells using a barcoded vector using Example 2. The library included about 3,546 variants. 10× scRNA-seq libraries were prepared using the HEK293 cells. Each of the variants were linked to variant barcodes by amplicon sequencing of cDNA derived from the HEK293 cells. The variants were linked to the cell barcode by interrogating the amplicon data with the scRNA-seq data. Example 2 provides illustrative, nonlimiting examples of methods of cloning a library of DNA into 5′UTR barcoded lentiviral vector: it will be appreciated that other suitable methods may be used.

Additional Comments

The practice of the present disclosure may employ, unless otherwise indicated, conventional techniques of molecular biology (including recombinant techniques), microbiology, cell biology, biochemistry and immunology, which are within the skill of the art. Such techniques are explained fully in the literature, such as, Molecular Cloning: A Laboratory Manual, 2^{nd} ed. (Sambrook et al., 1989): Oligonucleotide Synthesis (M. J. Gait, ed., 1984); Animal Cell Culture (R. I. Freshney, ed., 1987); Methods in Enzymology (Academic Press, Inc.); Current Protocols in Molecular Biology (F. M. Ausubel et al., eds., 1987, and periodic updates); PCR: The Polymerase Chain Reaction (Mullis et al., eds., 1994); Remington, The Science and Practice of Pharmacy, 20^{th} ed., (Lippincott, Williams & Wilkins 2003), and Remington, The Science and Practice of Pharmacy, 22^{th} ed., (Pharmaceutical Press and Philadelphia College of Pharmacy at University of the Sciences 2012).

All publications, patents, and patent applications mentioned in this specification are herein incorporated by reference to the same extent as if each individual publication, patent, or patent application was specifically and individually indicated to be incorporated by reference.

While various illustrative examples are described above, it will be apparent to one skilled in the art that various changes and modifications may be made therein without departing from the invention. The appended claims are intended to cover all such changes and modifications that fall within the true spirit and scope of the invention.

It is to be understood that any respective features/examples of each of the aspects of the disclosure as described herein may be implemented together in any appropriate combination, and that any features/examples from any one or more of these aspects may be implemented together with any of the features of the other aspect(s) as described herein in any appropriate combination to achieve the benefits as described herein.

1. A method of analyzing expression of a protein-coding region of DNA in a cell, the method comprising:
replacing a protein-coding region of the DNA in the cell with a donor vector comprising a variant of the protein-coding region and a first barcode identifying that variant,
wherein the cell generates mRNA comprising an expression of the variant and an expression of the first barcode;
coupling, to the mRNA, a second barcode corresponding to the cell;
reverse transcribing the mRNA, having the second barcode coupled thereto, into cDNA;
sequencing the cDNA;
sequencing the donor vector or cDNA using amplicon sequencing; and
correlating the donor vector sequence and the cDNA sequence to identify the variant and the cell's expression of the variant.

2. The method of claim 1, wherein the donor vector comprises a promoter region.

3. The method of claim 2, wherein the barcode is located between the promoter region and the variant.

4. The method of claim 2, wherein the donor vector comprises right and left homology arms, the variant and the first barcode being between the right and left homology arms.

5. The method of claim 2, wherein the promoter region comprises a reverse promotor region.

6. The method of claim 5, wherein the reverse promoter region is disposed between the first barcode and the variant.

7. The method of claim 5, wherein the expression of the variant of the protein-coding region is in the forward direction, and wherein the expression of the first barcode is in the reverse direction.

8. The method of claim 4, further comprising:
using a first polymerase chain reaction (PCR) process to generate a first amplicon of the donor sequence that includes the variant, the first barcode, and the right homology arm and substantially excludes the left homology arm; and
using a second PCR process to generate a second amplicon of the first amplicon that includes the variant and the first barcode and substantially excludes the right and left homology arms.

9. The method of claim 8, wherein sequencing the donor vector comprises sequencing the second amplicon.

10. The method of claim 8, wherein the second amplicon has a length of about 1000 bases or fewer.

11. The method of claim 1, wherein the mRNA comprises:
a first mRNA molecule comprising the expression of the variant, and
a second mRNA molecule comprising the expression of the first barcode.

12. The method of claim 11, wherein coupling the second barcode to the mRNA comprises:
coupling a first molecule of the second barcode to the first mRNA molecule; and
coupling a second molecule of the second barcode to the second mRNA molecule.

13. The method of claim 12, wherein the cDNA comprises a first cDNA molecule comprising a reverse transcription of the variant and the second barcode, and a second cDNA molecule comprising a reverse transcription of the protein coding region and the second barcode, and sequencing the cDNA comprises sequencing the first and second cDNA molecules.

14. The method of claim 1, wherein replacing the initial protein-coding region comprises:
using a CRISPR-associated protein guide RNA ribonucleoprotein (Cas-gRNA RNP) to cut the DNA in the cell; and
using homology-directed repair (HDR) to repair the cut in the DNA using the donor vector.

15. The method of claim 14, further comprising inserting first and second plasmids into the cell,
wherein the donor vector is located on the first plasmid; and
wherein the cell expresses the Cas-gRNA RNP using the second plasmid.

16. The method of claim 1, wherein the donor vector comprises a lentiviral vector.

17. The method of claim 1, wherein the donor vector further comprises a puromycin resistance gene, the method further comprising contacting the cell with puromycin to enrich for the cell.

18. The method of claim 17, wherein the first barcode is located on a UTR terminus of the puromycin resistance gene.

19. The method of claim 1, further comprising cleaving the first barcode from the variant in the cell.

20. A method of analyzing expression of a protein-coding region of DNA in a collection of cells, the method comprising:
replacing the initial protein coding-region of the DNA in each of the cells with a donor vector comprising a variant of the protein-coding region and a first barcode identifying that variant, wherein the cells receive different variants than one another;
obtaining mRNA from the cells, the mRNA from each cell comprising an expression of the variant of the protein-coding region in that cell and an expression of the first barcode;
coupling, to the mRNA from each cell, a second barcode corresponding to that cell;
reverse transcribing the mRNA, having the second barcode coupled thereto, into cDNA;
sequencing the cDNA;
sequencing the donor vector; and
correlating the donor vector sequence and the cDNA sequence to identify the variant in each of the cells and that cell's expression of that variant.

21. The method of claim 20, wherein the different variants are saturationally mutagenized.

22-23. (canceled)

24. A collection of polynucleotides from a collection of cells, the polynucleotides comprising first and second mRNA molecules from each of the cells, wherein, for each cell:
the first mRNA molecule comprises a first molecule of a barcode corresponding to that cell and an expression of a variant in that cell, and
the second mRNA molecule comprises the barcode corresponding to that cell and an expression of a first barcode corresponding to the variant.

25. The collection of polynucleotides of claim 24, wherein the different variants are saturationally mutagenized.

26. A method, comprising:
providing a barcoded homology donor vector comprising a semi-random barcode on termini of a foreign transcript, the donor vector including homology arms and mutations;
knocking-in the barcoded homology donor vector to the vicinity of an exon to be edited to create a variant on the exon; and
cleaving the variant using a CRISPR-associated protein guide RNA ribonucleoprotein (Cas-gRNA RNP).

27. The method of claim 26, wherein the barcode is placed on UTR termini of the donor vector so that it may be expressed and detectable in scRNA-seq.

28. The method of claim 26, wherein the donor vector comprises a puromycin resistance gene.

29. The method of claim 26, wherein providing the barcoded homology donor vector comprises:
using a first polymerase chain reaction (PCR) to specifically amplify the knocked-in region with a genomically edited allele;
using a second PCR, using the product of the first PCR as a template, to link the barcode with variants in an amplicon; and
performing amplicon sequencing using the product from the second PCR.

30. The method of claim 27, wherein the amplicon sequencing covers both the barcode and the variants.

31. A method, comprising:
adding semi-random variant barcodes to UTR regions of a saturationally mutagenized variant library;
coupling cell barcodes to the variant barcodes;
reading the variant barcodes out in scRNA-seq; and
linking the variant barcodes to the variants of the library using a separate sequencing operation.

32. The method of claim 31, wherein the semi-random variant barcode may be placed downstream of promoters or upstream of terminators of the variant library.

33. The method of claim 31, wherein linking the variant barcodes to the variants of the library comprises generating tiled polymerase chain reaction (PCR) amplicons by using one set of primers to amplify the barcode on one side, and another set of primers to amplify the variants on the other side, such that each amplicon links a respective segment of the variant to the barcode.

34-36. (canceled)
